(19) United States
(12) Patent Application Publication
(10) Pub. No.: US 2025/0345469 A1
(43) Pub. Date: Nov. 13, 2025

(54) COMPOSITION COMPRISING A RAPALOG AND A RADIOLABELLED GASTRIN ANALOGUE, IN PARTICULAR FOR USE IN THE TREATMENT AND/OR DIAGNOSIS OF CCKB RECEPTOR POSITIVE CANCER OR TUMORS

(71) Applicant: Paul Scherrer Institut, Villigen PSI (CH)

(72) Inventors: Michal Grzmil, Basel (CH); Roger Schibli, Baden (CH); Martin Behe, Gelterkinden (CH); Alain Blanc, Aarau (CH); Yun Qin, Ennetbaden (CH)

(21) Appl. No.: 18/040,011

(22) PCT Filed: Jul. 30, 2021
(86) PCT No.: PCT/EP2021/071422

§ 371 Date: Jan. 31, 2023

(30) Foreign Application Priority Data
Jul. 31, 2020 (WO) PCT/EP2020/071730

(51) Int. Cl. A61K 51/08; A61K 31/436; A61K 31/675; A61P 35/00

(52) U.S. Cl. CPC A61K 51/088; A61K 31/436; A61K 31/675; A61P 35/00

(57) ABSTRACT

A composition contains (i) rapamycin and/or a rapalog and (ii) a radiolabeled gastrin analogue. The composition can be used for the treatment and/or diagnosis of CCKB receptor positive cancer or tumors and leads to superior tumor uptake of radiolabeled gastrin analogue, resulting in improved delivery and therapeutic efficacy while cytotoxic side-effects are prevented and/or reduced.

### REFERENCE TO THE SEQUENCE LISTING

This application includes an electronically submitted sequence listing in .txt format. The .txt file contains a sequence listing entitled “2022P20057_ST25.txt” created on Aug. 3, 2023 and is 5,378 bytes in size. The sequence listing contained in this .txt file is part of the specification and is hereby incorporated by reference herein in its entirety.

### SPECIFICATION

The present invention relates to a composition comprising rapamycin and/or a rapalog and a radiolabeled gastrin analogue for the treatment and/or diagnosis of disease. In particular, the present invention relates to a composition for peptide receptor radionuclide therapy (PRRT) applications, which leads to superior tumor uptake of radiolabeled gastrin analogue via inhibition of mammalian target of rapamycin (mTOR), resulting in improved delivery and therapeutic efficacy while cytotoxic side-effects can be prevented and/or reduced.

### BACKGROUND OF THE INVENTION

G-protein coupled receptors (GPCRs) constitute a superfamily of membrane proteins whose function is to transduce a chemical signal across the cell membrane. When a ligand binds to a GPCR, it causes a conformational change allowing the GPCR to activate and release associated G proteins, which subsequently triggers signal transduction pathways.

Overexpression of G-protein coupled receptors (GPCRs) that selectively bind their peptide ligands allow the development of peptide receptor radionuclide therapy (PRRT) for human cancers (Lappano et al. Nat Rev Drug Discov. 2011, 10(1), 47-60). One of the most important goal of PRRT is to achieve high tumor uptake of radiolabeled ligands. Therefore, strategies to increase the uptake of radiopharmaceuticals in tumors or cancer tissue while sparing healthy organs from cytotoxic side effects have been considered. GPCRs targeted by agonistic ligand-based therapeutics undergo conformational changes, which lead to the exchange of GDP for GTP on the G-protein alpha subunit (Gα). Subsequent dissociation of the Gα and Gβγ subunits from the receptor results in activation of various kinase signaling pathways involving protein kinases A and C (PKA; PKC) as well as phosphoinositide 3-kinase (PI3K) and mitogen activated protein kinases (MAPKs) (O'Hayre et al. Curr Opin Cell Biol. 2014, 27, 126-135). Subsequently, activated GPCRs undergo desensitization via an arrestin-mediated internalization process, whereby GPCRs can be trafficked to lysosomes for degradation, or to endosomes for their recycling back to the cell surface (Rajagopal et al. Cell Signal. 2018, 41, 9-16). This internalization process enables the delivery of ligand-conjugated radioactive nuclides into target cells, e.g. cancer cells.

Medullary thyroid cancer (MTC) is a neuroendocrine tumor derived from calcitonin-producing C cells. Accounting for 3-5% of all thyroid cancers, MTC is a relatively rare cancer entity (Hadoux et al. Lancet Diabetes Endocrinol. 2016, 4(1), 64-71). Unfortunately, responses to conventional chemotherapy (usually doxorubicin alone or in combination with cisplatin) are only transient and benefit is limited to a small number of patients. In addition, MTC cells do not accumulate iodine and thus, do not respond to radioactive iodine treatment (Verburg et al. Methods. 201, 55(3), 230-237). Currently, MTC accounts for 14% of all thyroid cancer-related deaths, indicating the need for better treatments especially in metastasized patients (Roman et al. Cancer. 2006, 107(9), 2134-2142).

High expression of cholecystokinin B receptor (CCKBR, sometimes also referred to as CCK2R), which belongs to the GPCR family, has been validated in a variety of cancers including MTC, gliomas, as well as colon cancer, ovarian cancer etc. (Reubi et al. Cancer Res. 1997, 57(7), 1377-1386). Furthermore, the small peptide hormone minigastrin is known to bind to CCKBR with high affinity. Therefore, previous studies have suggested the use of radiolabeled gastrin analogues for PRRT, in particular for “theranostics” (therapy and diagnostics) applications (Behr et al. Semin Nucl Med. 2002, 32(2), 97-109; Kolenc-Peitl et al. J Med Chem. 2011, 54(8), 2602-2609; Laverman et al. Eur J Nucl Med Mol Imaging. 2011, 38(8), 1410-1416).

WO 2015/067473 A1 discloses a gastrin analogue having the formula DOTA-(DGlu)₆-Ala-Tyr-Gly-Trp-Nle-Asp-Phe-NH₂ (PP-F11N) (SEQ ID NO:10) which is radiolabeled with ¹⁷⁷Lu. This radiolabeled gastrin analogue (in the following “¹⁷⁷Lu-PP-F11N”) is chemically stable (e.g. resistant to proteolysis) and exhibits high tumor uptake as well as low accumulation in the kidneys. However, in a recent study, it has been found that ¹⁷⁷Lu-PP-F11N can accumulate in healthy tissues including stomach and colon due to their endogenous CCKBR expression (Sauter et al. J Nucl Med. 2019, 60(3), 393-399).

In view of the foregoing, it is an object of the present invention to provide a composition (kit-of-parts) for PRRT applications, which achieves superior uptake of radiolabeled gastrin analogues in CCKBR positive cancer or tumors, thus leading to improved delivery and therapeutic efficacy while the cytotoxic side-effects due to accumulation in heathy tissues can be prevented and/or reduced.

A further object of the present invention is to provide compositions (kit-of-parts) that can be used in methods of treating and/or diagnosing CCKBR positive cancer or tumors.

### SUMMARY OF THE PRESENT INVENTION

The present invention provides a composition (kit-of-parts, combination product) which can be used in PRRT applications, in particular in methods of treating and/or diagnosing CCKBR positive cancer or tumors such as MTC or gliomas. The present inventors have found that the (pre)treatment of CCKBR-expressing tumor cells with rapamycin and/or a rapalog, such as Everolimus (RAD001), leads to superior uptake of radiolabeled gastrin analog in these tumor cells, both in vitro and in vivo, resulting in improved delivery and therapeutic efficacy.

Furthermore, it has been surprisingly found that in vivo superior uptake of radiolabeled gastrin analogue specifically occurred in tumor cells but not in healthy tissues such as the gastrointestinal tract which endogenously express CCKBR. As a result, the side effects which can arise due to specific, or unspecific, accumulation of radiolabeled gastrin analogue in healthy tissues are prevented and/or reduced.

The present invention thus relates to a composition, a kit-of-parts, and a combination product comprising:
(i) rapamycin and/or a rapalog; and
(ii) a radiolabeled gastrin analogue.

The present invention also relates to a composition, kit-of-parts, and combination product as hereinbefore described for use in a method of treating and/or diagnosing CCKBR positive cancer or tumors, in particular MTC, gliomas, gastroenteropancreatic neuroendocrine tumors (GEP-NETs), astrocytomas, stomach cancer, colon cancer, ovarian cancer, breast cancer, and any CCKBR positive cancer and tumors.

The present invention in particular includes the following embodiments (“Items”):
1. Composition comprising:
   (i) rapamycin and/or a rapalog; and
   (ii) a radiolabeled gastrin analogue.
2. The composition according to item 1, wherein the rapalog is a compound selected from the group consisting of Everolimus (RAD001), Temsirolimus (CCI-779), Ridaforolimus (AP-23573), and combinations thereof, preferably Everolimus (RAD001).
3. The composition according to item 1 or 2, wherein the radiolabeled gastrin analogue has the following formula (1):

(1)
(SEQ ID NO: 1)
Y-S-Axx-Bxx-Cxx-Gly-Trp-Dxx-Asp-Phe-NH₂

wherein
   Axx represents an amino acid selected from Glu, D-Glu, Ala, Asp, Gln, Lys, His, Arg, Ser and Asn, preferably Glu or D-Glu;
   Bxx represents an amino acid selected from Ala, Ser, Val, Cys, Thr and Gly, preferably Ala or Ser;
   Cxx represents an amino acid selected from Tyr, Ala, Phe, Trp and His, preferably Tyr;
   Dxx represents an amino acid isosteric with methionine, preferably an amino acid selected from norleucine, 2-amino-5-heptenoic acid, homo-norleucine (homo-Nle), 2-amino-4-methoxybutanoic acid, telluro-methionine (Te-Met), seleno-methionine (Se-Met) and phenylglycine (Phg), more preferably Nle or Phg;
   S is a spacer comprising at least one selected from an amino acid containing a negative charge, Gln and D-Gln, preferably a tetra- or pentapeptide comprising at least one amino acid selected from Glu, D-Glu, beta-glutamic acid (beta-Glu), Gln, D-Gln, Asp and D-Asp, more preferably a pentapeptide comprising at least one amino acid selected from Glu, D-Glu, beta-Glu, Gln, D-Gln, Asp and D-Asp; and
   Y represents a moiety that comprises a radionuclide, e.g. a moiety that chelates a radionuclide or covalently bonds a radionuclide.
4. The composition according to any of items 1 to 3, wherein the radiolabeled gastrin analogue has the following formula (2):

(2)
(SEQ ID NO: 9)
Y-DGlu-DGlu-DGlu-DGlu-DGlu-DGlu-Ala-Tyr-Gly-Trp-
Dxx-Asp-Phe-NH₂

wherein Dxx represents an amino acid isosteric with methionine, preferably an amino acid selected from Nle, 2-amino-5-heptenoic acid, homo-Nle, 2-amino-4-methoxybutanoic acid, Te-Me, Se-Met and Phg, and Y represents a moiety that comprises a radionuclide, e.g. a moiety that chelates a radionuclide such as a radiometal, or covalently bonds a radionuclide.
5. The composition according to any of items 1 to 4, wherein Dxx is Nle and/or Y is 1,4,7,10-tetraazacyclododecane-1,4,7,10-tetraacetic acid (DOTA), 1,4,7-triazacyclononane-1,4,7-triacetic acid (NOTA), or 1,4,7-triazacyclononane,1-glutaric acid-4,7-acetic acid (NODAGA), preferably NODAGA.
6. The composition according to any of items 1 to 5, wherein the radionuclide is selected from the group consisting of ¹⁸F, ¹²⁴I, ¹³¹I, ⁸⁶Y, ⁹⁰Y, ¹⁷⁷Lu, ¹¹¹In, ¹⁸⁸Re, ⁶⁴Cu, ⁶⁷Cu, ¹⁴⁹Tb, ¹⁶¹Tb, ⁸⁹Sr, ^{44/43}Sc, ⁴⁷Sc and ¹⁵³Sm.
7. The composition according to any of items 1 to 6, wherein the radionuclide is selected from the group consisting of ¹⁷⁷Lu, ⁹⁰Y and ¹¹¹In.
8. The composition according to any of items 1 to 7, wherein the radionuclide is ¹⁷⁷Lu.
9. The composition according to any of items 1 to 8, wherein the rapalog is Everolimus (RAD001) and the radiolabeled gastrin analogue has the following formula (3):

(3)
(SEQ ID NO: 10)
DOTA-DGlu-DGlu-DGlu-DGlu-DGlu-DGlu-Ala-Tyr-Gly-
Trp-Nle-Asp-Phe-NH₂

wherein DOTA chelates ¹⁷⁷Lu.
10. The composition according to any of items 1 to 9, wherein the (i) rapamycin and/or rapalog and (ii) the radiolabelled gastrin analogue are formulated in separated dosage forms, which may be administered simultaneously and/or sequentially, preferably sequentially, and are co-packaged, or co-presented in separate packaging.
11. Kit-of-parts comprising:
   (i) rapamycin and/or a rapalog; and
   (ii) a radiolabeled gastrin analogue.
12. Kit-of-parts according to item 11, wherein (i) the rapalog and/or the (ii) radiolabelled gastrin analogue is/are as defined in any of items 2 to 9.
13. Combination product comprising:
   (i) rapamycin and/or a rapalog; and
   (ii) a radiolabeled gastrin analogue.
14. The combination product according to item 13, wherein (i) the rapalog and/or (ii) the radiolabelled gastrin analogue is/are as defined in any of items 2 to 9.
15. Composition according to any of items 1 to 10, kit-of-parts according to kit of part claims, or combination product according to item 13 or 14 for use in the treatment of CCKB receptor positive cancer or tumors, wherein the (i) rapamycin and/or rapalog is administered with (ii) the radiolabeled gastrin analogue, or is administered before (ii) the radiolabeled gastrin analogue.
16. The composition, kit-of-parts or combination product for the use according to item 15, wherein the (i) rapamycin and/or rapalog is administered up to two months before (ii) the radiolabeled gastrin analogue, preferably 1 to 14 days beforehand.
17. The composition, kit-of-parts or combination product for the use according to item 15 or 16, wherein the (i) rapamycin and/or rapalog is administered once daily over 1 to 14 consecutive days, preferably over 2 to 7 consecutive days, e.g. 5 consecutive days, before (ii) the radiolabeled gastrin analogue is administered.
18. The composition, kit-of-parts or combination product for the use according to any of items 15 to 17, wherein the CCKB receptor positive cancer or tumors is/are selected from medullary thyroid cancer (MTC), gliomas, gastroenteropancreatic neuroendocrine tumors (GEP-NETs), astrocytomas, stomach cancer, colon cancer, ovarian cancer, breast cancer, and any CCKB receptor positive tumors or cancer.
19. The composition, kit-of-parts or combination product for the use according to any of items 15 to 18, wherein the CCKB receptor positive cancer or tumors is MTC.
20. The composition, kit-of-parts or combination product for the use according to any of items 15 to 19, wherein the (i) rapamycin and/or rapalog or (ii) the radiolabeled gastrin analogue is administered concurrently with, before or after one or more other therapeutic agents or therapies such as chemotherapeutic agents or immunomodulatory agents.
21. Method for treating CCKB receptor positive cancer or tumors, wherein a therapeutically effective amount of the (i) rapamycin and/or rapalog and (ii) the radiolabeled gastrin analogue comprised in the composition according to any of items 1 to 10, kit-of-parts according to item 11 or 12, or combination product according to item 13 or 14 is administered to a patient in need thereof.

### FIGURES

FIG. 1—Identification of kinase inhibitors for enhancement of cellular uptake of ¹⁷⁷Lu-PP-F11N. (1A) Experimental design: Kinase inhibitor treated, and control untreated A431/CCKBR cells were subject to analysis of cellular uptake of ¹⁷⁷Lu-PP-F11N as described in the section “material and methods” below. (1B, C) Volcano plots represent changes in cellular uptake of ¹⁷⁷Lu-PP-F11N and proliferation shown as log 2 [ratio: treatment/control], respectively. Dots marked as BML-257, SC-514 and rapamycin represent kinase inhibitors that significantly increased uptake of ¹⁷⁷Lu-PP-F11N (P<0.05).

FIG. 2—Inhibition of mTORC1 activity increases internalization of ¹⁷⁷Lu-PP-F11N. (2A) Internalized and cell-bound activity was measured after 1 h incubation with ¹⁷⁷Lu-PP-F11N in control and 100 nM RAD001 and 10 mM metformin-treated A431/CCKBR, untransfected A431 (negative control) and AR42J cells for 20 h, as indicated. All experiments were assayed in triplicate. Bars represent mean±SD. **P<0.01, ***P<0.001. (2B) Mean protein concentration±SD of lysates used in C. Mean protein concertation in control cells were set to 1. (2C) Western blot analysis using phosho-specific S6 antibody on whole-cell lysates isolated from control and 20 h RAD001- or metformin-treated A431/CCKBR cells. Blots were stripped and re-probed with GAPDH antibody for loading control.

FIG. 3—RAD001-increased CCKBR expression is required for enhanced internalization of ¹⁷⁷Lu-PP-F11N. WB analysis using anti-CCKBR antibody of the glycosylated and deglycosylated (PNGase F-treated) lysates from A431/CCKBR cells, following 20 h RAD001 treatment (3A) or transfection (3B) with luciferase siRNA (control) and CCKBR siRNA for 48 h, as indicated. Blots were stripped and re-probed with GAPDH antibody for loading control. Below; quantification of the CCKBR signal intensities in deglycosylated lysates normalized to GAPDH. The CCKBR/GAPDH ratios from untreated or control transfected cells were set to 1.

FIG. 4—RAD001 increases CCKBR-dependent tumor uptake of ¹⁷⁷Lu-PP-F11N in vivo (5-day treatment). (4A) A431/CCKBR cells were implanted into immunocompromised (nude) mice. Five days after implantation RAD001, metformin and control (PBS) animal groups received daily dose as indicated by triangles and the biodistribution study was accomplished on the next day. Bars: biodistribution of ¹⁷⁷Lu-PP-F11N analyzed 4 h after tail vein administration shown as % of total injected radioactivity (% iActivity) per gram of tissue. Lower panel: Corresponding biodistribution after co-injection with a blocking peptide. (4B, C) Tumor volume and mouse weight before (1st Day) and after treatment (5^{th} day) in all groups. Bars represent mean±SD.

FIG. 5—RAD001 increases CCKBR-dependent tumor uptake of ¹⁷⁷Lu-PP-F11N in vivo (5-day treatment). (5A) SPECT/CT images 2 h after ¹⁷⁷Lu-PP-F11N injections of metformin, RAD001 and control (PBS) treated mice for 5 days. Below: corresponding radioactive tumor regions. (5B) Average and maximum activity concentration±SD of ¹⁷⁷Lu-PP-F11N in radioactive regions of 4 tumors per group. *P<0.05, **P<0.01, ***P<0.001.

FIGS. 6 and 7—RAD001 increases CCKBR-dependent tumor uptake of ¹⁷⁷Lu-PP-F11N in vivo (3-day treatment). (6, 7A) A431/CCKBR cells were implanted into immunocompromised nude mice. RAD001, metformin and control (PBS) animal groups received 3 daily dose and the biodistribution study was accomplished on the next day. Bars: biodistribution of ¹⁷⁷Lu-PP-F11N analyzed 4 h after tail vein administration shown as % of total injected radioactivity (% iActivity) per gram of tissue. (7B) Tumor volume and mouse weight before and after 3-day treatment. Data represent mean±SD. *P<0.05.

FIGS. 8 and 9—Increased necrosis and reduced number of mitotic figures and Ki67 positive cells in RAD001-treated tumors. Paraffin sections prepared from A431/CCKBR-tumors treated with RAD001, Metformin (Met) and PBS (control) were subjected to HE and Ki67 staining as described in material and methods. Bars represent percent of necrotic area (8A), no. of vessels per field (8B), mitotic index (9A) and percent of Ki67 positive cells (9B) shown as an average±SD of analyzed tumor groups. Right; images from representative HE and Ki67 staining. Arrows in 8B and 9A indicate vessels, and mitotic figures, respectively. Scale bar 0.5 mm in 8A and 20 μm in 8B and 9AB. *P<0.05, **P<0.01, ***P<0.001.

FIGS. 10, 11 and 12—Tumor growth inhibition and prolonged life span in RAD001 and ¹⁷⁷Lu-PP-F11N-treated mice. (10A) Experimental design: After implantation of A431/CCKBR cells into nude mice, 5 or 10 doses of RAD001 was administrated alone or in combination with 60 kBq ¹⁷⁷Lu-PP-F11N, as indicated. (10B) The tumor growth curves of control and treated groups. (FIG. 11) Tumor volume 13, 22 and 25 days after beginning of the treatment. Data represent mean±SD. (FIG. 12) The survival rates presented as Kaplan-Meier curves of the control and treated groups.

### DETAILED DESCRIPTION OF THE PRESENT INVENTION

### 1. Definitions

The term “composition” as used herein is to be understood as referring to a combination of individual components (i.e. to a combination product), namely (i) the rapalog and (ii) the radiolabeled gastrin analogue, which are kept physically separate from each other but adjacent. Such compositions are sometimes referred to as “kit-of-parts”. In some aspects, the term “composition” as used herein encompasses the physical mixture of individual components, for instance if the (i) rapamycin and/or rapalog or (ii) the radiolabeled gastrin analogue is to be used concurrently with one or more other therapeutic agents or therapies, provided that the (i) rapamycin and/or rapalog and (ii) the radiolabeled gastrin analog are kept physically separate from each other, e.g. are formulated in separate dosage forms.

The term “rapamycin” (Sirolimus, Rapamune®) refers to a macrolide compound, which is known in the art to exhibit immunosuppressant properties by inhibiting the mammalian target of rapamycin (mTOR). Rapamycin has the following chemical structure:

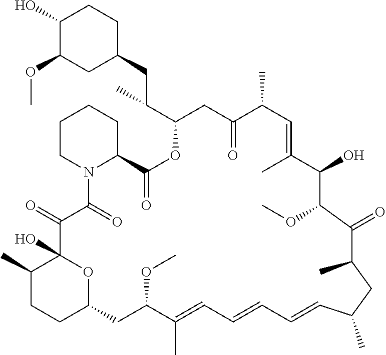 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

The term “rapalog” (which stands for “rapamycin-analog”) as used herein refers to a class of compounds structurally related to rapamycin, which are known to inhibit the mammalian target of rapamycin in complex 1 (mTORC1) by binding to the FK-binding protein 12 (MacKeigan et al. Neuro-Onc. 2015, 17(12), 1550-1559). Examples of rapalogs include Everolimus (RAD001, Afinitor®, Certican®), Temserolimus (CCI-779, Torisel®) and Ridaforolimus (AP-23573, MK-8669).

The inhibitor activity of the rapamycin and/or rapalog towards mTORC1 can be determined by measuring the level of phosphorylation of ribosomal protein S6 by Western Blot analysis as described further below.

The expression “gastrin analogue” as used herein refers to a class of compounds (peptides) structurally related to the endogenous peptide hormone gastrin, which can bind to the CCKBR. The expression “gastrin analogue” as used herein is meant to encompass all compounds containing the C-terminal amino acid sequence Gly-Trp-Dxx-Asp-Phe-NH₂ (SEQ ID NO:11), wherein Dxx is Met or an amino acid isosteric with Met, as found in CCKBR-binding endogenous peptide hormones including e.g. gastrin and cholecystokinin (CCK). Gastrin is a linear peptide hormone produced by G cells of the duodenum and in the pyloric antrum of the stomach. It is secreted into the bloodstream. The encoded polypeptide is pre-progastrin, which is cleaved by enzymes in posttranslational modification to produce progastrin and then gastrin in various forms, including primarily big-gastrin (G-34), little gastrin (G-17), and minigastrin (Leu-Glu-Glu-Glu-Glu-Glu-Ala-Tyr-Gly-Trp-Met-Asp-Phe-NH₂)(SEQ ID NO:2) which all represent “gastrin analogues” in the sense of the present invention. CCK is a peptide hormone structurally related to gastrin in that both compounds share five C-terminal amino acids i.e. Gly-Trp-Met-Asp-Phe-NH₂ (SEQ ID NO:3)(wherein Met can be replaced by an amino acid isosteric with Met such as norleucine). CCK exists naturally in several forms including e.g. CCK8 (Asp-Tyr-Met-Gly-Trp-Met-Asp-Phe-NH₂)(SEQ ID NO:4). The gastrin analogue can be chemically modified, e.g. at its N-terminus, for covalent attachment to a spacer or a moiety that is able to chelate radiometals, such as 1,4,7,10-tetraazacyclododecane-1,4,7,10-tetraacetic acid (DOTA), or a moiety that (covalently) bonds a radionuclide such as ¹⁸F or iodine isotopes. In some instances, the gastrin analogue can be modified for covalent attachment to an imaging moiety for medical applications such as Alexa Fluor® 647, IRDye 680RD or DY-700, or to a photosensitizer such as Photofrin, Forscam or Photochlor.

The expression “gastrin analog” also refers to gastrin, big-gastrin, little gastrin, CCK, CCK8 or minigastrin, in particular minigastrin, wherein one, two or more amino acid residues are replaced for another natural or unnatural amino acid residue, provided that the resulting “gastrin analog” remains pharmacologically active with respect to CCKBR. In some aspects of the present invention, said modification may allow achieving e.g. enhanced binding affinity and/or pharmacological activity towards CCKBR, enhanced plasma stability, enhanced biodistribution or the like. Pharmacological activity in this connection means that the gastrin analogue retains at least 20%, preferably at least 50%, more preferably at least 80% of the pharmacological (agonistic) activity of minigastrin.

The pharmacological activity of the gastrin analogue towards CCKBR can be determined by measuring the intracellular increase of calcitonin level in gastrin analogue-stimulated cells as described by Bläker et al. (Regulatory Peptides 2004, 118, 111-117).

In some instances, the expression “gastrin analogue” refers to a compound (peptide) that is structurally related to the compound having the formula (DGlu)₆-Ala-Tyr-Gly-Trp-X-Asp-Phe-NH₂ (SEQ ID NO:8) wherein X represents an amino acid isosteric with methionine. The compound can be modified for covalent attachment to a spacer or a moiety that can chelate a radionuclide (e.g. radiometal), such as DOTA, or a moiety that (covalently) bonds radionuclides such as ¹⁸F or iodine isotopes. When X represents norleucine and said compound is modified at its N-terminus by covalent attachment of DOTA, the compound corresponds to “PP-F11N”.

The term “amino acid” as used herein refers to a compound that contains or is derived from at least one amino group and at least one acidic group, preferably a carboxyl group. The distance between amino group and acidic group is not particularly limited. α-, β-, and γ-amino acids are suitable but α-amino acids and especially α-amino carboxylic acids are particularly preferred. This term encompasses both naturally occurring amino acids as well as synthetic amino acids that are not found in nature.

The expression “(D)-amino acid” as used herein refers to the (D)-isomer of any naturally occurring or synthetic amino acid. For instance, the expression “D-Glu” refers to the (D)-isomer of glutamic acid. The expression “(D)-amino acid” as used herein is not meant to encompass non-chiral amino acids such as glycine or other non-chiral amino acids.

The expression “amino acid Isosteric with methionine” as used herein refers to a natural or unnatural amino acid having a shape and/or electronic properties similar to those of methionine. The term “isosteric” as used herein is meant to encompass amino acids, which are essentially isosteric of methionine such as norleucine (Nle). Examples of amino acids isosteric with methionine include Nle, 2-amino-5-heptenoic acid, homo-norleucine (homo-Nle), 2-amino-4-methoxybutanoic acid, telluro-methionine (Te-Met), seleno-methionine (Se-Met), and phenylglycine (Phg).

Unless specified otherwise or dictated otherwise by the context, all connections between adjacent amino acid groups are formed by peptide (amide) bonds.

The expression “moiety that chelates or (covalently) bonds a radionuclide” as used herein refers to a moiety (chelating agent or ligand) that can either (i) donate electrons to a radionuclide, in particular radiometal, to form a coordination complex therewith, i.e. by forming at least one coordinate covalent bond (dipolar bond) therewith, or (ii) covalently bond a radionuclide such as ¹⁸F or iodine isotopes. The chelating mechanism depends on the chelating agent and/or radionuclide. For example, it is believed that DOTA can coordinate a radionuclide via carboxylate and amino groups (donor groups) thus forming complexes having high stability (Dai et al. Nature Corn. 2018, 9, 857).

The expression “amino acid containing a negative charge” (or “negatively charged amino acid”) is used herein to characterize natural or unnatural amino acids, wherein the side chain contains an ionizable group (in solution) bearing a negative charge, in particular a carboxylic acid group. Examples of natural or unnatural amino acids containing a negative charge include Glu, D-Glu, beta-glutamic acid (beta-Glu), Asp, D-Asp, 2-amino-adipic acid, 2-amino-pimelic acid, and 2-amino-suberic acid.

The term “cancer” as used herein means the pathological condition in mammalian tissues that is characterized by abnormal cell growth to form malignant tumors, which may have the potential to invade or spread to other tissues or parts of the body to form “secondary” tumors known as metastases. A tumor comprises one or more cancer cells. The expression “CCKBR positive cancer or tumors” as used herein refer to cancer or tumors that are characterized by overexpression of the CCKBR on the cell surface (Reubi et al. Cancer Res. 1997, 57(7), 1377-1386). Examples of CCKBR positive cancer or tumors include MTC, gliomas, gastroenteropancreatic neuroendocrine tumors (GEP-NETs), astrocytomas, stomach cancer, colon cancer, ovarian cancer and breast cancer.

The term “Internalization” as used herein refers to the biological process in which molecules (e.g. a radiolabeled gastrin analogue) are engulfed by the cell membrane and drawn into the cell. As a result, the molecules (e.g. the radiolabeled gastrin analogue) are present inside the cell.

The expression “cell uptake” (of radiopharmaceuticals) refers to the biological process in which molecules (e.g. a radiolabeled gastrin analogue) are internalized and/or bound on the cell membrane. As a result, the molecules (e.g. the radiolabeled gastrin analogue) can be present inside the cell as well as at the cell membrane.

The expression “tumor uptake” (of radiopharmaceuticals) refers to the biological process in which molecules (e.g. a radiolabeled gastrin analogue) are taken up by tumor (cancer) cells. Tumor uptake includes tumor cell uptake of molecules (e.g. the radiolabeled gastrin analogue) and/or the retention thereof in the tumor microenvironment. As a result, the molecules (e.g. the radiolabeled gastrin analogue) can be present inside the tumor (cancer) cell, at the cell membrane and/or within the tumor microenvironment.

The expression “co-administration” as used herein refers to the concurrent (simultaneous) administration of both the (i) rapamycin and/or rapalog and (ii) the radiolabeled gastrin analogue as well as the sequential administration of the (i) rapamycin and/or rapalog and, subsequently, (ii) the radiolabeled gastrin analogue. Moreover, the expression “co-administration” is meant to encompass dosing schedules wherein the respective dosages of (i) rapamycin and/or a rapalog and/or (ii) a radiolabeled gastrin analogue are varied (increased or decreased) during the course of the administration. Preferably, the (i) rapamycin and/or rapalog and (ii) the radiolabeled gastrin analogue are administered in a sequential pattern according to which the (i) rapamycin and/or rapalog is first administered over a period of time and, subsequently, the (ii) radiolabeled gastrin analogue is administered.

Where the present description refers to “preferred” embodiments/features, combinations of these “preferred” embodiments/features shall also be deemed as disclosed as long as this combination of “preferred” embodiments/features is technically meaningful.

Hereinafter, in the present description of the invention and the claims, the use of the terms “containing”, “Including” and “comprising” is to be understood such that additional unmentioned elements may be present in addition to the mentioned elements. However, these terms should also be understood as disclosing, as a more restricted embodiment, the term “consisting of” as well, such that no additional unmentioned elements may be present, as long as this is technically meaningful.

Unless the context dictates otherwise and/or alternative meanings are explicitly provided herein, all terms are intended to have meanings generally accepted in the art, as reflected by IUPAC Gold Book (status of 1 Nov. 2017), or the Dictionary of Chemistry, Oxford, 6^{th} Ed.

### 2. Overview

The present invention is based on the discovery that the (pre)treatment of CCKBR-expressing tumor cells with rapamycin and/or a rapalog such as Everolimus (RAD001) leads to a significant increase of CCKBR protein level and to superior uptake of radiolabeled gastrin analog, resulting in improved delivery and therapeutic efficacy while cytotoxic side effects due to accumulation in healthy tissues are prevented and/or reduced.

One of the most important goal for efficient PRRT is a high tumor uptake of the radioactive compound, which largely depends on the expression level and internalization-related activity of the targeted receptor. The present inventors have surprisingly identified a group of kinase inhibitors which can enhance the uptake of radiolabeled gastrin analogs in CCKBR positive tumor cells (e.g. A431/CCKBR cells). In particular, it has been found that the (pre)treatment of CCKBR positive tumor cells with a rapalog (e.g. RAD001) led to a significant increase of CCKBR level and subsequently to increased CCKBR-specific internalization of radiolabeled gastrin analogue, resulting in improved delivery and therapeutic efficacy.

Furthermore, the enhanced CCKBR-specific uptake of radiolabeled gastrin analogue was observed in tumor cells but not in healthy organs such as gastrointestinal tract which endogenously expresses CCKBR, indicating that rapalog-treatment leads to superior tumor cell targeting. As a result, the cytotoxic side effects which can arise due accumulation of the radiolabeled gastrin analogue in healthy tissues can be prevented and/or reduced.

Without being bound to any theory, it is believed that tumor cells exhibit higher mTORC1 activity as compared to healthy tissues, thereby leading to superior efficacy in tumor cells while accumulation thereof in healthy tissues is prevented and/or reduced.

### 3. Composition

The composition (kit-of-parts, combination product) of the present invention is provided in the form of a pharmaceutical composition (formulation) for human or animal usage in human and veterinary medicine. Typically, (i) the rapamycin and/or rapalog and (ii) the radiolabeled gastrin analogue are presented as separate pharmaceutical compositions for co-administration (kit-of-parts), wherein each respective composition comprises a therapeutically effective amount of (i) rapamycin and/or a rapalog and (ii) a radiolabeled gastrin analogue.

In some aspects of the present invention, the individual components (i) and (ii) are formulated in separated dosage forms, which may be co-packaged or co-presented in separate packaging. In some instances, the packaged component (ii) may include an unlabeled gastrin analog, which can be labeled with a radionuclide shortly prior to administration, e.g. by chelating a radionuclide as described further below.

The composition (or each part of the kit) can additionally comprise one or more components selected from a carrier, a diluent and other excipients. Suitable carriers, diluents and other excipients for use in pharmaceutical compositions are well known in the art, and are for instance described in Remington's Pharmaceutical Sciences, Mack Publishing Co. (Gennaro A R, 1985). The carrier, diluent and/or other excipient can be selected with regard to the intended route of administration and pharmaceutical practice. The composition can comprise as the carrier, diluents and/or other excipients, or in addition to, any suitable binder(s), lubricant(s), suspending agent(s), coating agent(s), solubilizing agent(s).

The therapeutically effective amount of (i) rapamycin and/or rapalog and/or (ii) radiolabeled gastrin analogue can be determined by a physician on a routine basis. The specific dose level and frequency of dosage for any particular subject/patient can vary and depends on a variety of factors including the activity of the specific drug compound employed, the metabolic stability and length of action of that compound, the patient's age, body weight, general health, sex, diet, mode and time of administration, rate of excretion, drug combination, the severity of the particular condition, and the individual undergoing therapy. These factors are considered by the physician when determining the therapeutically effective dose.

The rapalog as used herein is not particularly limited provided that it exhibits mTORC1 inhibitory activity and leads to enhanced CCKBR level on the cell surface. The level of CCKBR on the cell surface can be determined by methods known in the art, e.g. by Western Blot analysis, as described further below. The mTORC1 inhibitor activity of the rapamycin and/or rapalog can be determined by measuring the phosphorylation level of ribosomal protein S6 by Western Blot analysis as described further below.

According to one embodiment, the rapalog is selected from the group consisting of Everolimus (RAD001), Temsirolimus (CCI-779), Ridaforolimus (AP-23573), and combinations thereof. According to one preferred embodiment, the rapalog is Everolimus (RAD001). Some of these compounds are commercially available under the trade names Rapamune® (Sirolimus), Afinitor® or Certican® (Everolimus), Torisel® (Temserolimus).

For instance, a rapalog such as Everolimus (RAD001) can be administered, e.g. orally, in daily dosages ranging from 0.1 mg to 20 mg, such as 0.1 mg to 15 mg, preferably from 0.5 mg to 10 mg e.g. 5 mg in the form of (dispersible) tablets comprising the rapalog, such as Everolimus, e.g. as a solid dispersion.

The radiolabeled gastrin analogue as used herein is not particularly limited provided that it binds CCKBR and shows agonistic activity therefor. In one embodiment, the gastrin analogue is represented by the following formula (1):

(1)
(SEQ ID NO: 1)
Y-S-Axx-Bxx-Cxx-Gly-Trp-Dxx-Asp-Phe-NH₂

wherein,
Axx represents an amino acid selected from Glu, D-Glu, Ala, Asp, Gln, Lys, His, Arg, Ser and Asn, preferably Glu or D-Glu;
Bxx represents an amino acid selected from Ala, Ser, Val, Cys, Thr and Gly, preferably Ala or Ser;
Cxx represents an amino acid selected from Tyr, Ala, Phe, Trp and His, preferably Tyr;
Dxx represents an amino acid isosteric with methionine, preferably an amino acid selected from norleucine, 2-amino-5-heptenoic acid, homo-norleucine (homo-Nle), 2-amino-4-methoxybutanoic acid, telluro-methionine (Te-Met) and seleno-methionine (Se-Met), more preferably Nle;
S is a spacer comprising at least one selected from
   an amino acid containing a negative charge,
   Gln, and
   D-Gln;
preferably S is a tetra- or pentapeptide comprising at least one amino acid selected from Glu, D-Glu, beta-glutamic acid (beta-Glu), Gln, D-Gln, Asp and D-Asp, more preferably a pentapeptide comprising at least one amino acid selected Glu, D-Glu, beta-Glu, Gln, D-Gln, Asp and D-Asp; and
Y represents a moiety that comprises a radionuclide, e.g. a moiety that chelates a radionuclide (e.g. radiometal), or a moiety that (covalently) bonds a radionuclide. Preferably, Y represents a moiety that chelates a radionuclide.

In one preferred embodiment, the gastrin analogue is represented by the following formula (2):

(2)
(SEQ ID NO: 9)
Y-DGlu-DGlu-DGlu-DGlu-DGlu-DGlu-Ala-Tyr-Gly-Trp-
Dxx-Asp-Phe-NH₂

wherein X represents an amino acid isosteric with methionine such as norleucine (Nle), 2-amino-5-heptenoic acid, homo-norleucine (homo-Nle), 2-amino-4-methoxybutanoic acid, telluro-methionine (Te-Met), seleno-methionine (Se-Met) or Phg, and Y represents a moiety that comprises a radionuclide, e.g. a moiety that chelates a radionuclide (e.g. radiometal), such as DOTA, NOTA, or NODAGA, preferably NODAGA, or a moiety that (covalently) bonds a radionuclide.

In some aspects, Y (as defined in formulae (1) and (2)) includes a functional group such as a carboxylic acid for covalent attachment to the peptide, i.e. for covalent attachment to the N-terminus of the peptidic chain or for covalent attachment to the side chain of the N-terminal amino acid.

Examples of moieties which can chelate a radionuclide (chelators) include diethylenetriaminepentaacetic acid (DTPA), desferoxamine (DFO), 1,4,7-triazacyclononane,1-glutaric acid-4,7-acetic acid (NODAGA), 1,4,7,10-tetraazacyclododecane-1-glutaric acid-4,7,10-triacetic acid (DOTAGA), 2,2′-(1,4,7-triazacyclononane-1,4-diyl)diacetate (NO2A), 1,4,7,10-tetraatacyclododecane-1,4,7,10-tetraacetic acid (DOTA), 1,4,7-triazacyclononane-1,4,7-triacetic acid (NOTA), ethylenediaminetetraacetic acid (EDTA), ethylenediaminediacetic acid, triethylenetetraminehexaacetic acid (TTHA), 1,4,8,11-tetraazacyclotetradecane (CYCLAM), 1,4,8,11-tetraazacyclotetradecane-1,4,8,11-tetraacetic acid (TETA), 1,4,8,11-tetraazabicyclo[6.6.2]hexadecane-4,11-diaceticacid (CB-TE2A), 2,2′,2″-(1,4,7,10-tetraazacyclododecane-1,4,7-triyl)triacetamide (DO3AM), 1,4,7,10-tetraazacyclododecane-1,7-diacetic acid (DO2A), 1,5,9-triazacyclododecane (TACD), (3a1s,5a1s)-dodecahydro-3a,5a,8a,10a-tetraazapyrene (cis-glyoxal-cyclam), 1,4,7-triazacyclononane (TACN), 1,4,7,10-tetraazacyclododecane (cyclen), tri(hydroxypyridinone) (THP), 3-(((4,7-bis((hydroxy(hydroxymethyl)phosphoryl)methyl)-1,4,7-triazonan-1-yl)methyl)(hydroxy)phosphoryl)propanoic acid (NOPO), 3,6,9,15-tetraazabicyclo[9.3.1]pentadeca-1(15),11,13-triene-3,6,9-triacetic acid (PCTA), 2,2′,2″,2″′-(1,4,7,10-tetraazacyclotridecane-1,4,7,10-tetrayl)tetraacetic acid (TRITA), 2,2′,2″,2″′-(1,4,7,10-tetraazacyclotridecane-1,4,7,10-tetrayl)tetraacetamide (TRITAM), 2,2′,2″-(1,4,7,10-tetraazacyclotridecane-1,4,7-triyl)triacetamide (TRITRAM), trans-N-dimethyl-cyclam, 2,2′,2″-(1,4,7-triazacyclononane-1,4,7-triyl)triacetamide (NOTAM), oxocyclam, dioxocyclam, 1,7-dioxa-4,10-diazacyclododecane, cross-bridged-cyclam (CB-cyclam), triazacyclononane phosphinate (TRAP), dipyridoxyl diphosphate (DPDP), meso-tetra-(4-sulfanotophenyl)porphine (TPPS₄), ethylenebishydroxyphenylglycine (EHPG), hexamethylenediaminetetraacetic acid, dimethylphosphinomethane (DMPE), methylenediphosphoric acid, dimercaptosuccinic acid (DMPA), and derivatives thereof.

In one embodiment, X represents an amino acid isosteric with methionine selected from Nle, 2-amino-4-methoxybutanoic acid, Te-Met, Se-Met, 2-amino-5-heptenoic acid, homo-Nle and Phg, preferably Nle, and/or Y represents a moiety selected from DOTA, DTPA, NOTA, NODAGA, and TETA, preferably DOTA, NOTA or NODAGA, more preferably NODAGA. According to one embodiment, X represents Nle and Y represents DOTA.

The gastrin analogue of formula (1) or (2) exhibits excellent resistance to degradation by proteases and is stable in systemic circulation. Moreover, the gastrin analogue of formula (1) or (2) also shows very low uptake and accumulation in the kidneys, and hence side effects due to unspecific accumulation in healthy tissues (e.g. kidneys) can be reduced even further.

In one preferred embodiment, the radiolabeled gastrin analogue is represented by the following formula (3):

(3)
(SEQ ID NO: 10)
DOTA-DGlu-DGlu-DGlu-DGlu-DGlu-DGlu-Ala-Tyr-Gly-
Trp-Nle-Asp-Phe-NH₂

wherein DOTA chelates a radionuclide, which is preferably ¹⁷⁷Lu.

The radionuclide as used herein can be selected from any naturally occurring or artificially produced atom having excess nuclear energy, which emits beta and/or gamma radiation. In one embodiment, the radionuclide is selected from the group consisting of ¹⁸F, ¹²⁴I, ¹³¹I, ⁸⁶Y, ⁹⁰Y, ¹⁷⁷Lu, ¹¹¹In, ¹⁸⁸Re, ⁶⁴Cu, ⁶⁷Cu, ¹⁴⁹Tb, ¹⁶¹Tb, ⁸⁹Sr, ^{44/43}Sc, ⁴⁷Sc and ¹⁵³Sm.

According to one preferred embodiment, the radionuclide is selected from the group consisting of ¹⁷⁷Lu, ⁹⁰Y and ¹¹¹In, and more preferably is ¹⁷⁷Lu.

### 4. Use of Composition in Methods of Treating and/or Diagnosing Diseases

The composition (kit-of-parts) of the present invention including (i) rapamycin and/or a rapalog and (ii) a radiolabeled gastrin analogue can be used to treat and/or diagnose disease, in particular to treat and/or diagnose CCKBR positive cancer or tumors. In some aspects, the treatment can prolong survival of a subject as compared to expected survival of the subject if not receiving the treatment.

The disease that is treated by the composition (kit-of-parts) can be any neoplastic disease such as cancer characterized by the expression of CCKBR. Non-limiting examples of CCKBR positive cancer or tumors include medullary thyroid cancer (MTC), gliomas, gastroenteropancreatic neuroendocrine tumors (GEP-NETs), astrocytomas, stomach cancer, colon cancer, ovarian cancer, and breast cancer. An exemplary disease is MTC.

According to one embodiment, the composition of the present invention is used in a method of treating or diagnosing CCKBR positive cancer or tumors, by co-administering the (i) rapamycin and/or rapalog and (ii) the radiolabeled gastrin analogue to a patient in need thereof. One or more rapalogs and one or more radiolabeled gastrin analogues can be co-administered in all aspects of the present invention. Co-administration of the rapalog with the radiolabeled gastrin analogue comprises the concurrent (simultaneous) administration of both the (i) rapamycin and/or rapalog and (ii) the radiolabeled gastrin analogue as well as the sequential administration of the (i) rapamycin and/or rapalog and, subsequently, (ii) the radiolabeled gastrin analogue. If there is more than one rapalog, these can be administered either individually each on its own and/or together as a rapamycin/rapalog cocktail. Similarly, if there is more than one radiolabeled gastrin analogue, these can be again administered individually each on its own and/or together as a cocktail.

Without being bound to any theory, it is believed that co-administration allows sufficient exposure of CCKBR positive tumor cells to the rapalog to inhibit the mTOR pathway and to achieve the increase in CCKBR density on the cell surface prior to exposure of the same tumor cells to the radiolabeled gastrin analogue thereby achieving superior targeting of the radiolabeled gastrin analogue to the tumor cells. Therefore, co-administration comprises any mode of administering a rapalog in conjunction with a radiolabeled gastrin analogue that achieves this result.

The rapamycin and/or rapalog and the radiolabeled gastrin analogue can be administered concurrently (simultaneously) or independently from each other, e.g. according to a sequential administration pattern wherein (i) an mTOR inhibitor, in particular rapamycin and/or the rapalog is administered over a predetermined period of time before (ii) the radiolabeled gastrin analogue.

According to one embodiment, the (i) rapamycin and/or rapalog is administered simultaneously with (ii) the radiolabeled gastrin analogue or before (ii) the radiolabeled gastrin analogue. Thus, the (i) rapamycin and/or rapalog can be administered on the same day as (ii) the radiolabeled gastrin analogue, either together or within hours of each other but can also be administered up to about two months beforehand.

According to one preferred embodiment, the (i) rapamycin and/or rapalog is/are first administered before (ii) the radiolabeled gastrin analogue. Dosing schedules can be adjusted to fit the patent needs and disease state (progression) as well as the rapalog and radiolabeled gastrin analogue. The objective pursued by the dosing schedule is to administer component (i) so as to increase CCKBR expression on the surface of CCKBR positive tumor cells thereby improving targeting and uptake of the radiolabeled gastrin analogue.

Preferably the rapamycin and/or rapalog is/are administered prior to administration of (ii) the radiolabeled gastrin analogue over a period of up to two months, preferably 1 to 14 days beforehand, e.g. over a period of 2 to 5 consecutive days such as 3 days. In particular, the component (i) can be administered once or several times daily, preferably once daily, over a period of 1 to 14 consecutive days, preferably over 2 to 7 consecutive days, for instance over 3 or 5 consecutive days, before (ii) the radiolabeled gastrin analogue is administered.

Furthermore, co-administration includes administering more than one dose of (ii) radiolabeled gastrin analogue within several weeks after one or more doses of rapalog. Thus, the rapamycin and/or rapalog need not be re-administered with every subsequent administration of the radiolabeled gastrin analogue but can be administered just once, or intermittently during the course of the radiolabeled gastrin analogue treatment.

The rapalog can be administered, e.g. orally, in daily dosages ranging from 0.1 mg to 20 mg, such as 0.1 mg to 15 mg, preferably from 0.5 mg to 10 mg e.g. 5 mg in the form of (dispersible) tablets comprising the rapalog e.g. as a solid dispersion.

The radiolabeled gastrin analogue can be administered once or several times daily.

In one embodiment, the composition of the present invention can be administered concurrently with, before or after one or more other therapeutic agents or therapies such as chemotherapeutic agents and/or immunomodulatory agents. Examples of therapeutic agents or therapies that can be used include antineoplastic agents such as alkylating agents, alkaloids or kinase inhibitors, immunomodulatory agents and pharmaceutically acceptable salts and derivatives thereof.

### 5. Preparation of the Radiolabeled Gastrin Analogue

In the following, methods are provided for the preparation of the radiolabeled gastrin analogue. The gastrin analogue can be synthesized relying on standard Fmoc-based solid-phase peptide synthesis (SPPS), including on-resin peptide coupling and convergent strategies. The general strategies and methodology which can be used for preparing and radiolabeling the gastrin analogue of the present invention are well-known to the skilled person and also described further below.

### 6. Examples

### 6.1 List of Abbreviations

DIEA: diisopropylethylamine
DMEM: Dulbecco's Modified Eagle Medium
DMF: dimethyl formamide
DMSO: dimethyl sulfoxide
DTT: dithiothreitol
ESI: electron spray ionization
FCS: fetal calf serum
HATU: 1-[Bis(dimethylamino)methylene]-1H-1,2,3-triazolo[4,5-b]pyridinium 3-oxide hexafluorophosphate
HBTU: 3-[Bis(dimethylamino)methyliumyl]-3H-benzotriazol-1-oxide hexafluorophosphate
HPLC: high-performance liquid chromatography
IU: international unit
PBS: phosphate-buffered saline
RPMI: Roswell Park Memorial Institute
SOD: single quadrupole detection
SPECT: single-photon emission computed tomography
SPPS: solid-phase peptide synthesis
TBST: tris-buffered saline with Tween 20
TFA: trifluoroacetic acid
TIS: triisopropylsilane
UPLC: ultra-performance liquid chromatography

### 6.2 Materials and Methods

The following materials and methods were used to evaluate the composition of the present invention.

### 6.2.1 Cell Culture, Transfection and Treatments

Human epidermoid carcinoma A431 stable cell line that overexpress CCKBR, generated as previously described (Aloj et al., J Nucl Med 2004, 45(3), 485-94), was cultured in DMEM, whereas the rat pancreatic acinar cell line AR42J was grown in RPMI medium, supplemented with 10% FCS, 2 mM glutamine and antibiotics (0.1 mg/mL streptomycin, 100 IU penicillin) at 37° C. and 5% CO₂.

For CCKBR-specific knock-down, duplex siRNAs against CCKBR or control duplex against luciferase (Microsynth) were used at a final concentration of 100 nM in Optimem (Gibco): CCKBRseq1 sense RNA 5′-UAUACGAGUAGUAGCACCAdTdT-3′(SEQ ID NO:5), CCKBRseq2 sense RNA 5′-CCGCCAAAGGAUGGAGUACdTdT-3′(SEQ ID NO:6) and control sense RNA 5′-CGUACGCGGAAUACUUCGAdTdT-3′(SEQ ID NO:7). Cells at 60-80% confluence were transfected with siRNAs by using Lipofectamin 3000 according to the user's manual. Total protein lysates were subjected to WB analysis.

Treatment with RAD001 or metformin (both from Selleckchem) diluted in DMSO or water, respectively, was performed at indicated time points and PBS-washed cells were used for analyses described below.

### 6.2.2 Preparation of Gastrin Analogues

The gastrin analogues described herein were prepared by standard Fmoc-based SPPS, including on-resin peptide coupling and convergent strategies using an Activo-P-11 Automated Peptide Synthesizer (Activotec) and a Rink Amide resin (loading: 0.60 mmol/g; Novabiochem).

Coupling reactions for amide bond formation were performed over 30 min at room temperature using 3 eq of Fmoc-amino-acids activated with HBTU (2.9 eq) in the presence of DIEA (6 eq.). Fmoc deprotection was conducted with a solution of 20% piperidine in DMF. Coupling of the N-terminal labeling moiety can be performed over 30 min at room temperature using 3 eq of DOTA tris-t-Bu ester (Novabiochem) activated with HATU (2.9 eq) in the presence of DIEA (6 eq).

The peptides were cleaved from the resin under simultaneous side-chain deprotection by treatment with TFA/TIS/water (95/2.5/2.5, v/v/v) during 60 min. After concentration of the cleavage mixture, the crude peptides were precipitated with cold diethyl ether and centrifugated.

The peptides were purified on a Waters Autopurification HPLC system coupled to SOD mass spectrometer with a XSelect Peptide CSH C18 OBD Prep column (130 Å, 5 μm, 19 mm×150 mm) using solvent system (0.1% TFA in water) and B (0.1% TFA in acetonitrile) at a flow rate of 25 mL/min and a 20-60% gradient of B over 30 min. The appropriate fractions were associated, concentrated and lyophilized. The purity was determined on a Waters Acquity UPLC System coupled to SOD mass spectrometer with CSH C18 column (130 Å, 1.7 μm, 2.1 mm×50 mm) using solvent system A (0.1% TFA in water) and (0.1% TFA in acetonitrile) at a flow rate of 0.6 mL/min and a 5-85% gradient of B over 5 min.

MS-analysis was performed using electrospray ionization (ESI) interface in positive and negative mode.

### 6.2.3 Radiolabeling of Gastrin Analogues

A solution of N-terminal DOTA-conjugated gastrin analogue PP-F11N (DOTA-(DGlu)₆-Ala-Tyr-Gly-Trp-Nle-Asp-Phe) (SEQ ID NO:10) prepared as described above and ¹⁷⁷Lu (available from ITG GmbH) in a nuclide/peptide ratio of 1:30 was prepared in 0.4 M ammonium acetate buffer (pH 5.5) and the labeling was carried out at 90° C. for 15 min.

The lutetium incorporation was analyzed by standard HPLC using a C18 column and reached above 95% efficiency. Directly after labeling, a gamma counter was used to prepare appropriate dilutions of radiolabeled gastrin analogues for targeted radiation experiments.

### 6.2.4 Kinase Inhibitor Library Screen

25.000 cells per well were seeded on isoplate 96 TC (PerkinElmer) and subjected to treatment with 10 μM of kinase inhibitors (KIs) for 18 h (Screen-Well® Kinase Inhibitor Library, Enzo Life Sciences AG). On the next day, 10.000 cpm of ¹⁷⁷Lu-PP-F11N (prepared as described above) was added to each well and the plate was incubated for 2 h at standard tissue culture condition. The blocking control was performed at the presence of 4 μM of minigastrin LEEEEEAYGWMDF (SEQ ID NO:2) (PSL GmbH).

After removal of radioactive supernatant, cells were resuspended in 50 μl of ULTIMA GOLD high flash-point LSC-cocktail (Sigma) and incubated on a horizontal shaker for 2 h at RT. The activity was measured within 30 sec per well by using MicroBeta 2450 Microplate Counter (PerkinElmer). The screen was assayed in triplicates.

### 6.2.5 Proliferation Assay

Cells were seeded in 96-well plates before treatment as described above, and cell proliferation was analyzed by a CellTiter 96 AQueous Non-Radioactive Cell Proliferation Assay (Promega) according to the manufacturer's instructions. Absorbance of MTS bio-reduced into a formazan was measured at 570 nm with a reference of 650 nm using a MicroPlate Reader (PerkinElmer). The assay was performed in triplicate.

### 6.2.6 In Vitro Radiolabelled Peptide Internalization Assays

2.5×10⁵ cells were seeded on 6-well plates. Eight hours later cells were treated with appropriate inhibitors (as indicated) for 20 h. On the next day, PBS-washed cells were incubated with 100.000 cpm of ¹⁷⁷Lu-PP-F11N (in DMEM with 0.1% BSA) at standard TC condition. For blocking experiments, minigastrin (PSL GmbH) was used at 4 μM concentration. After 1 or 2 h incubation, radioactive medium (together with PBS wash) was collected and the cells were subjected to 2× wash with glycine buffer (pH=2) for 5 min at RT followed by a dissolving step in 1 M NaOH for 15 min at 37° C. All 3 collected fractions (medium/PBS; glycine wash; dissolved cells) were measured on a Cobra II Auto-Gamma counter (Packard); internalized and membrane-bound fractions of ¹⁷⁷Lu-PP-F11N are shown as % of total activity. Results from experiments with blocking peptides (control for unspecific binding or internalization in glycine wash or NaOH-dissolved cells, respectively) below 0.1% of total activity (data not shown) were subtracted from obtained results. Protein concentration of NaOH-dissolved cell lysates was measured by spectrophotometry using a NanoPhotometer® UV-Vis P-Class (Implen).

### 6.2.7 WB Analysis

Antibodies against phospho-S6 at S235/S236 (D57.2.2F) and GAPDH (14C10) were obtained from Cell Signaling Technology, whereas anti-CCKBR (ab77077) was from Abcam. Cells were homogenized in lysis buffer (50 mM Tris-HCl pH 7.5, 150 mM NaCl, 1% Triton X, 0.1% SDS supplemented with 1 mM sodium orthovanadate, 1 mM NaF and protease inhibitor cocktail (Roche)). Aliquots of 50 μg protein extracts were separated by SDS-PAGE and transferred to PVDF membranes (Millipore) by electroblotting. Membranes were blocked with 5% skim milk in TBST (0.1% Tween 20) for 1 h, and incubated with 2% BSA in TBST overnight with the primary antibody followed by 2 h incubation with HRP-conjugated secondary antibody. Protein-specific signals were detected by a chemiluminescence reagent (ECL) and signals were acquired by using ImageQuant RT ECL Imager (GE Healthcare).

### 6.2.8 Deglycosylation

Prior to CCKBR detection, protein lysates were subjected to deglycosylation. Briefly, 18 μL of whole cell lysate (approx. 50 μg) were mixed with 2 μL of 10× denaturing buffer (5% SDS, 0.4 M DTT) and incubated for 10 min at RT. Next, 4 μL of 10× Glycobuffer (0.5 M sodium phosphate, pH 7.5), 4 μL of 10% Tween-20 and 10 μL of water were added. Finally, 2 μL of PNGase F (Sigma) was added, mixed and collected by centrifugation. The reaction was carried out at 37° C. overnight before WB analysis.

### 6.2.9 Animal Study

In the study human A431/CCKBR xenograft mouse model was used. Importantly, immunodeficient mice bearing A431-CCK2R xenografts were previously used for the preclinical evaluation of radiolabeled minigastrin analogue pharmacokinetics, biodistribution, dosimetry or toxicity, required for regulatory approval of a phase I clinical trial in medullary thyroid cancer (MTC) patients (Maina et al., Eur J Pharm Sci. 2016; 91:236-42).

Immunocompromised CD-1 female nude mice (Charles Rivers, Germany) were housed one week before experimentation. Prior to tumor implantation, A431/CCKBR cells were harvested and 5 min cells in 0.1 mL of sterile phosphate-buffered saline (PBS) containing 0.9% NaCl were injected subcutaneously (two tumors per animal) into mice anesthetized by isoflurane/oxygen inhalation. After 5 days, animals were randomly distributed into 3 experimental groups and tumor size was measured non-invasively with a caliper. RAD001 (3 mg/kg), metformin (200 mg/kg) or PBS (control) were administered daily via intraperitoneal injection for 3 or 5 consecutive days, as indicated. During treatment cycles, no signs of acute toxicity were observed in any of the groups. After treatment, mice received ¹⁷⁷Lu-PP-F11N into the tail vein (approx. 150 kBq per mouse in a volume of 0.1 mL sterile PBS) and 4 h later mice were subjected to humane euthanasia with CO₂. Post mortem dissected tumors and organs were weighed and their activity was measured in a gamma counter. At termination, no significant differences in body or organ weights, general health, or anatomy were observed. All experiments were performed in accordance with Swiss Animal Protection Laws.

### 6.2.10 SPECT/CT

Prior to i.v. injections, ¹⁷⁷Lu-PP-F11N was purified by HPLC, concentrated on SpeedVac and diluted in PBS (10 MBq in 100 μl). Tumor uptake of ¹⁷⁷Lu-PP-F11N in A431/CCKBR-tumor bearing nude mice treated with metformin, RAD001 or PBS (control) was monitored by single-photon emission computed tomography (SPECT) combined with X-ray computed tomography (multipinhole small-animal NanoSPECT/CT camera, Mediso Medical Imaging Systems) according manufacturer's instruction. All mice were sacrificed 2 h after injections and used directly for 10 min CT followed by a 5 h SPECT scan. Image reconstruction, processing and relative quantification was accomplished by using VivoQuant 3.0 Patch1 software. Otsu method and connecting thresholding was applied for selection and analysis of the radioactive tumor regions.

### 6.2.11 Immunochemistry

Paraffin sections of formalin-fixed A431/CCKBR tumors were subjected to deparaffinization. Rehydrated slides were pretreated in 10 mM citrate buffer, pH 6.0, at 98° C. for 60 minutes, followed by incubation with 4% fat-free milk in PBS for 90 minutes. For avidin/biotin blocker treatment (Invitrogen) and detection, the ABC method was used according to the manufacturer's instructions. For monoclonal antibody against Ki67 (Thermo Scientific, SP6) signals were recorded using an automated instrument reagent system (Discovery XT, Ventana Medical System Inc.) according to the user manual. Images of hematoxylin-counterstained sections were captured (Nikon, YTHM) and analyzed using ImageAccess Enterprise7 and ImageJ software (Schneider et al. Nat Methods 2012, 9(7), 671-675).

### 6.2.12 Statistical Analysis

Two-tailed Student's t tests were performed for analysis of two groups, whereas one-way ANOVA followed by multiple comparison tests were performed for three or more groups using GraphPad Prism 7.00 for Windows. Values of P<0.05 were considered statistically significant.

### Example 1: Internalization of Radiolabeled Gastrin Analogue In Vitro—Kinase Inhibitor Library Screen

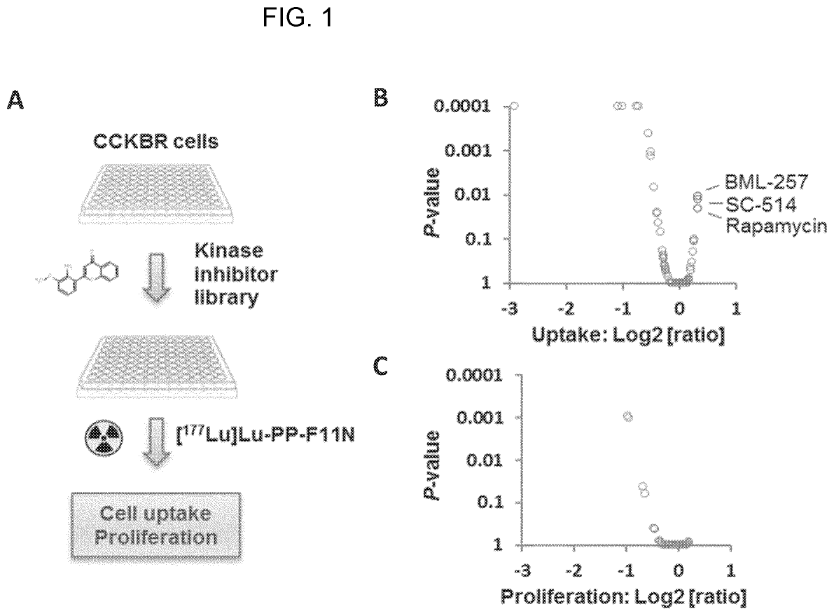

To investigate the pathways influencing the internalization rate of gastrin analogues, a library of kinase inhibitors (80 compounds) was screened in accordance with the procedure described above to determine ¹⁷⁷Lu-PP-F11N uptake in MTC A431/CCKBR cells. An overview of the screening assay is depicted in FIG. 1A.

Briefly, the cells were subjected to treatment with 10 μM of kinase inhibitors for 18 h. On the next day, 10.000 cpm of ¹⁷⁷Lu-PP-F11N (prepared as described above) was added to each well and the plate was incubated for 2 h at standard tissue culture condition. After removal of radioactive supernatant, the cells were resuspended, and incubated on a horizontal shaker for 2 h at RT. The activity was measured within 30 sec per well by using MicroBeta 2450 Microplate Counter.

It was found that 3 inhibitors, namely BML257, SC-514 and rapamycin, enhanced ¹⁷⁷Lu-PP-F11N uptake by 24-26% as compared to untreated controls (FIG. 1B). Matching proliferation assays did not show significant changes in cell proliferation for these three inhibitors indicating that the enhanced uptake did not result from increased cell numbers after kinase inhibition (FIG. 1C). BML-257 and rapamycin are known to target the AKT/mTORC1 pathway.

BML257 and SC-514 are kinase inhibitors having the following chemical structures:

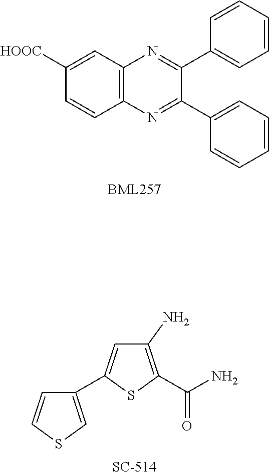 [SMILES: O=C(O)c1ccc2nc(-c3ccccc3)c(-c3ccccc3)nc2c1 ; NC(=O)c1sc(-c2ccsc2)cc1N]

### Example 2: RAD001- and Metformin-Induced Internalization of Radiolabeled Gastrin Analogue In Vitro

To further investigate the interference with AKT/mTORC1 signaling, the internalization of ¹⁷⁷Lu-PP-F11N in MTC A431/CCKBR cells was evaluated using the well-characterized and clinically approved mTORC1 inhibitors RAD001 (Everolimus) and metformin.

For the assay, the cells were treated with metformin or RAD001 for 20 hours and ¹⁷⁷Lu-PP-F11N internalization was determined in accordance with the procedure described above.

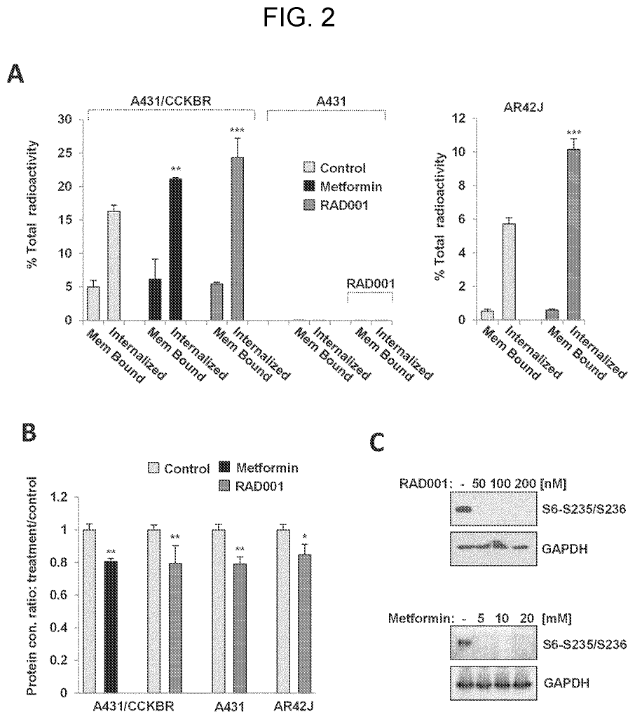

It was found that the treatment of the A431/CCKBR cells with metformin and RAD001 led to an increase of the ¹⁷⁷Lu-PP-F11N internalization of 21.1 and 24.3%, respectively, as compared to 16.2% for the control cells (ratio [treatment/control] of 1.3 and 1.5). Furthermore, the treatment of AR42J cells with RAD001 led to an increase of the ¹⁷⁷Lu-PP-F11N internalization of 10% as compared to 5.7% for the control cells with a ratio [RAD001/control] of 1.7. There was no significant difference in membrane bound activity in RAD001 or metformin treated cells (FIG. 2A). Protein levels in RAD001- and metformin-treated A431/CCKBR cells as well as RAD001-treated AR42J cells were reduced to 80, 79 and 84%, respectively, as compared to control (FIG. 2B).

The effects of RAD001 and metformin treatments were examined by WB analysis and showed lack of mTORC1-regulated phosphorylation of ribosomal protein S6 at Ser235/236 in protein lysates obtained from treated cells (FIG. 2C).

### Example 3: RAD001-Induced CCKBR Expression and ¹⁷⁷Lu-PP-F11N Internalization In Vitro

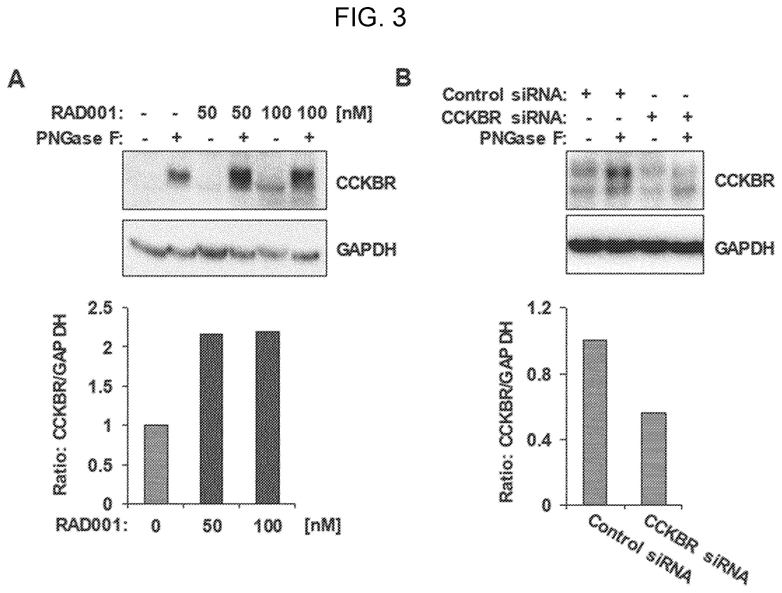

The expression of CCKBR in A431/CCKBR cells was evaluated in RAD001-treated cells. The results are depicted in FIG. 3A.

It was found that the treatment with either 50 nM or 100 nM RAD001 led to an increase of the CCKBR expression (approximately 2.2-fold), indicating that the increased internalization of radiolabeled gastrin analogue in RAD001-treated cells resulted from the elevated CCKBR expression. The detection of endogenous CCKBR was verified by RNA interference, whereby transfection with duplex siRNAs against CCKBR reduced CCKBR expression level to 55% as compared to control cells transfected with duplex siRNAs against the luciferase gene (FIG. 3B).

### Example 4: CCKBR-Specific Internalization of ¹⁷⁷Lu-PP-F11N in RAD001-Treated MTC Tumor In Vivo (5-Day Treatment)

To investigate the effects of RAD001 and metformin in vivo, A431/CCKBR cells were implanted subcutaneously into immunocompromised nude mice, and 5 days later after tumor formation, mice were subjected to treatment with RAD001, metformin or PBS (control) as described above.

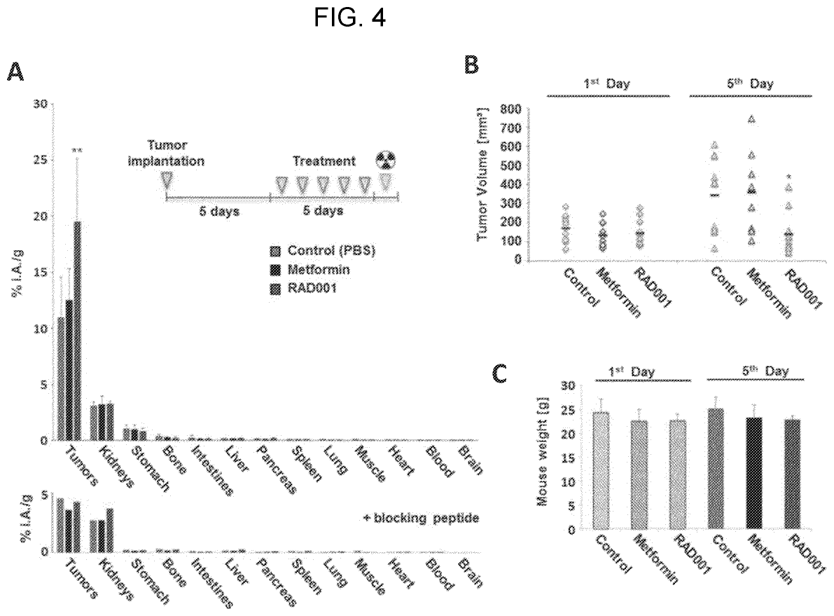

Biodistribution analysis showed a statistically significant increase (P=0.0057) in tumor uptake of ¹⁷⁷Lu-PPF-11N in RAD001-treated animals with a ratio [RAD001/Control] of 1.79 whereas, metformin treatment increased ¹⁷⁷Lu-PP-F11N internalization only moderately with a ratio [Metformin/Control] of 1.14 and did not reach statistical significance (FIG. 4A).

Furthermore, there was no significant difference in biodistribution of ¹⁷⁷Lu-PP-F11N in healthy organs in all groups. Co-injection of the radiolabeled gastrin analogues together with the unlabeled blocking peptide minigastrin (competitor for CCKBR binding) profoundly reduced ¹⁷⁷Lu-PP-F11N tumor uptake in all three groups demonstrating CCKBR-specific binding of radiolabeled gastrin analogues (FIG. 4A lower panel).

As shown in FIGS. 4B and 4C, the treatment with RAD001/¹⁷⁷Lu-PP-F11N significantly reduced tumor volumes whereas overall mouse weight was not affected. By contrast, the treatment with metformin/¹⁷⁷Lu-PP-F11N had no effect on tumor volume or mouse weight.

To further investigate whether the observed increased tumor uptake was influenced by the differences in the tumor masses between RAD001 and control group, statistical analysis was carried out in the groups of tumors with matching sizes. It was found that RAD001-treated tumors ranging from 50-150 or 50-180 mg showed statistically significant increase in ¹⁷⁷Lu-PP-F11N uptake without any significant differences in the tumor masses, indicating that RAD001 treatment increases tumor uptake of radiolabeled minigastrin in tumor-mass-independent manner (data not shown).

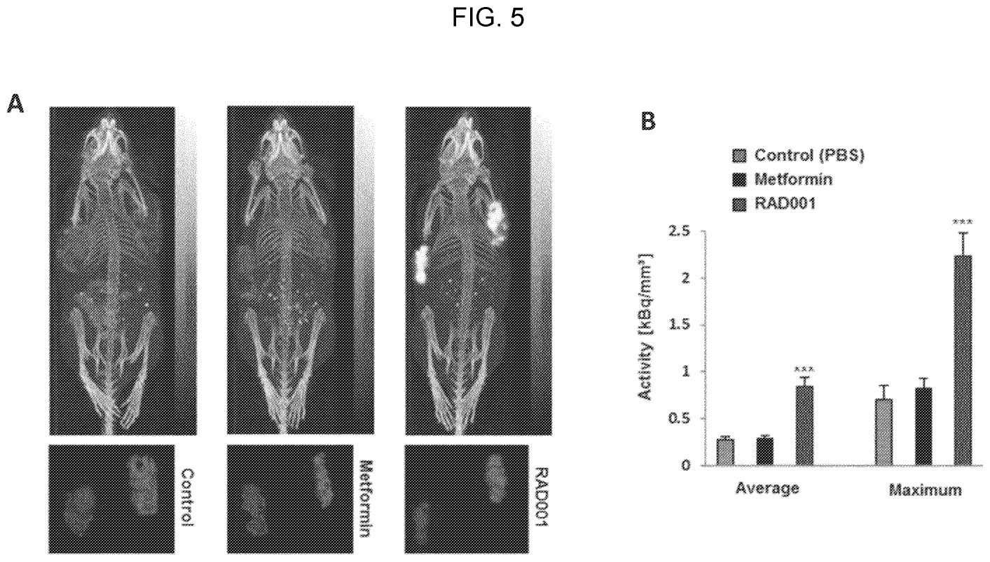

Further SPECT/CT imaging confirmed increased tumor uptake of ¹⁷⁷Lu-PP-F11N in A431/CCKBR-tumor bearing nude mice after 5 days treatment with RAD001 as compared to control (FIG. 5A). Relative quantification of the acquired images showed significantly higher (P<0.001) average and maximum concentration of radiolabeled minigastrin in RAD001-treated tumors as compared to control (FIG. 5B). Ratios [RAD001/Control] of average and maximum concentration reached 3.11 and 3.17, respectively, whereas metformin treatment did not show significant change with average and maximum concentration ratios [Metformin/Control] of 1.03 and 1.17, respectively.

### Example 5: CCKBR-Specific Internalization of ¹⁷⁷Lu-PP-F11N in RAD001-Treated CCKBR Tumor In Vivo (3-Day Treatment)

To investigate whether less than 5 doses of RAD001 would increase ¹⁷⁷Lu-PP-F11N tumor uptake, the same A431/CCKBR xenograft mouse model was subjected to 3-day treatment with RAD001 (3 doses), metformin or PBS (control).

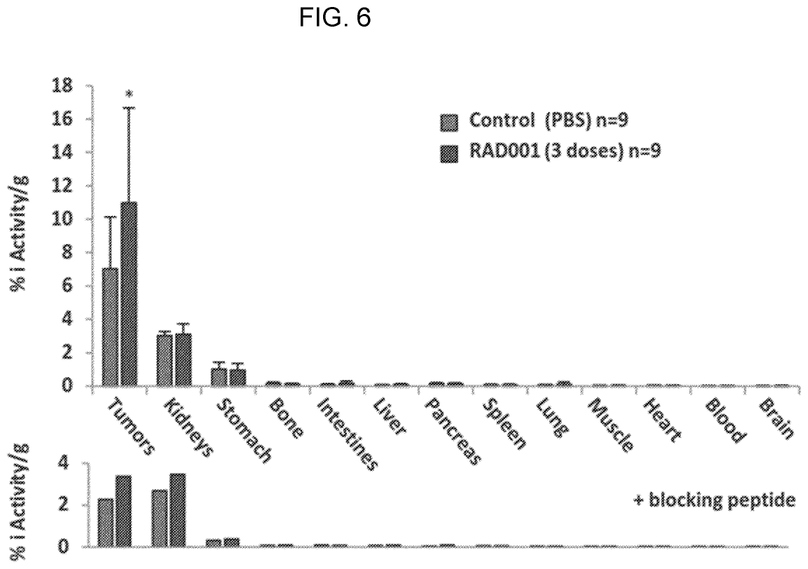

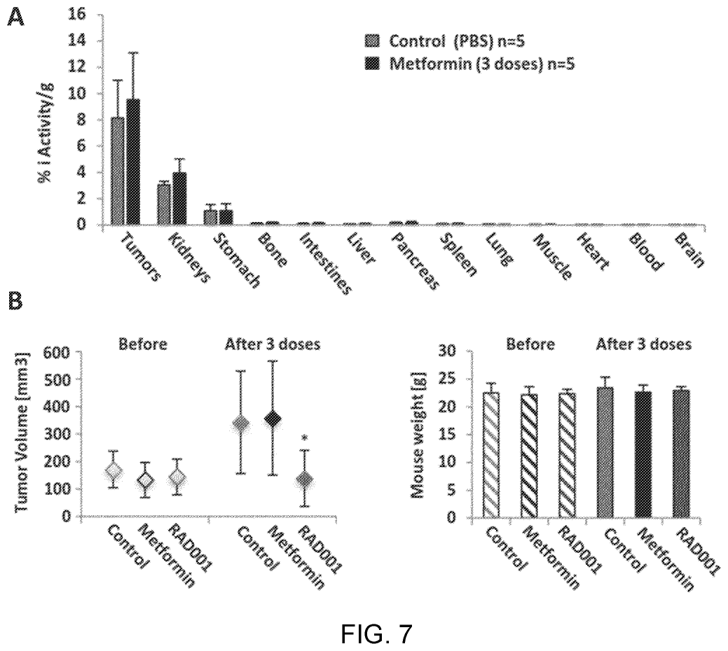

Biodistribution analysis demonstrates statistically significant increase (P=0.016) in tumor uptake of ¹⁷⁷Lu-PP-F11N in RAD001-treated animals with ratio [RAD001/Control] of 1.56 (FIG. 6) whereas, co-injection together with a blocking peptide (minigastrin) profoundly reduced ¹⁷⁷Lu-PP-F11N tumor uptake showing CCKBR-specific uptake. Three doses of metformin did not significantly influence ¹⁷⁷Lu-PP-F11N uptake (FIG. 7A).

There was no significant difference in biodistribution of ¹⁷⁷Lu-PP-F11N in healthy organs in all groups. Three daily doses of RAD001 significantly reduced tumor volumes without influencing overall mouse weight whereas, metformin treatment had no effect on tumor volume or mouse weight (FIG. 7B).

### Example 6: Necrosis and Number of Mitotic Figures and Ki67 Positive Cells in RAD001-Treated CCKBR Tumor In Vivo

Paraffin tumor sections prepared from A431/CCKBR-tumor bearing nude mice treated with five daily doses of RAD001, metformin and PBS (control) were subjected to immunohistochemistry. RAD001 and PBS group includes 5 tumors, while in metformin group 4 tumors were analyzed.

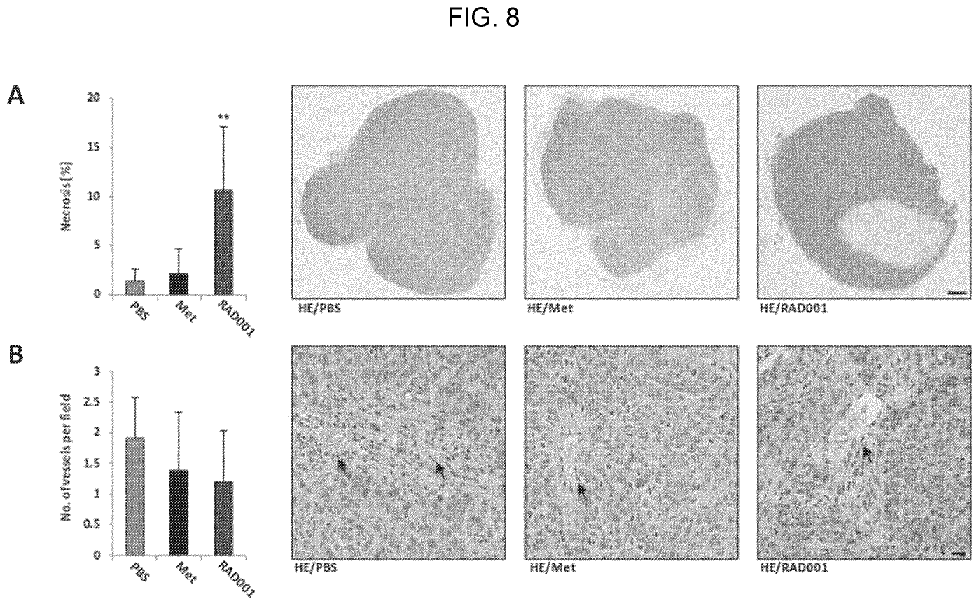

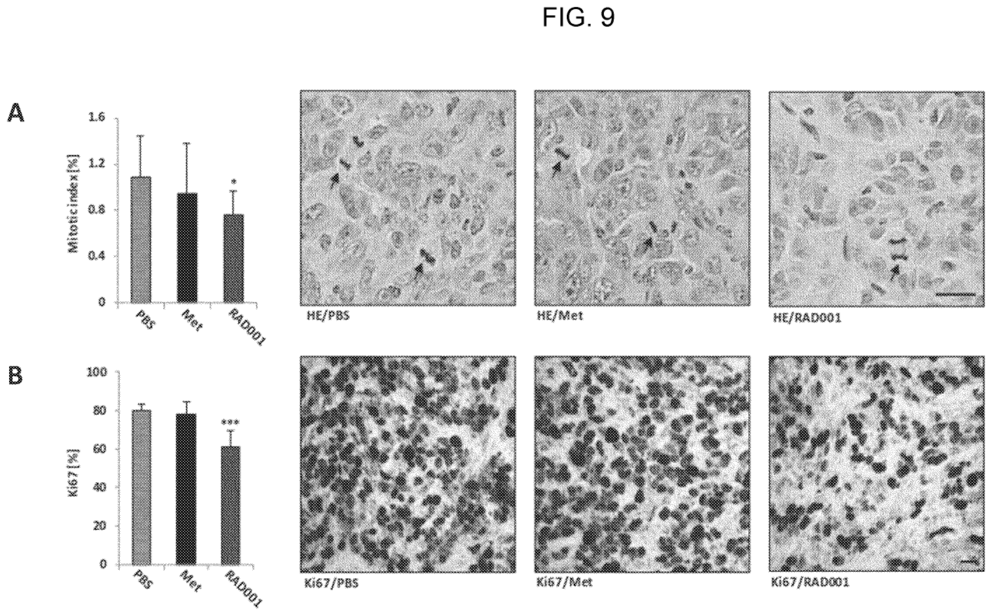

As shown in FIG. 8A, RAD001-treatment increased necrosis (10.6±6.4%) as compared to PBS-treated tumors (1.4±1.2%), whereas metformin did not show significant difference as compared to control. Analysis of vessel numbers showed no statistical difference between analyzed groups indicating that observed increased uptake of radiolabeled minigastrin in vivo is due to increased CCKBR expression in RAD001-preterted tumors (FIG. 8B). Reduced mitotic index (0.75±0.36%) and number of Ki67 positive cells (61±8.6%) was detected in RAD001-treated tumors as compared to control tumors that show 1.08±0.2% of mitotic index and 80±3.1% Ki67 positive cells (FIGS. 9A and 9B).

Metformin treatment did not cause significant change in the number of mitotic figures or Ki67 positive cells as compared to control.

### Example 7: Evaluation of Therapeutic Response to Combinatory Treatment with RAD001 and ¹⁷⁷Lu-PP-F11N in A431/CCKBR-Tumor Bearing Nude Mice

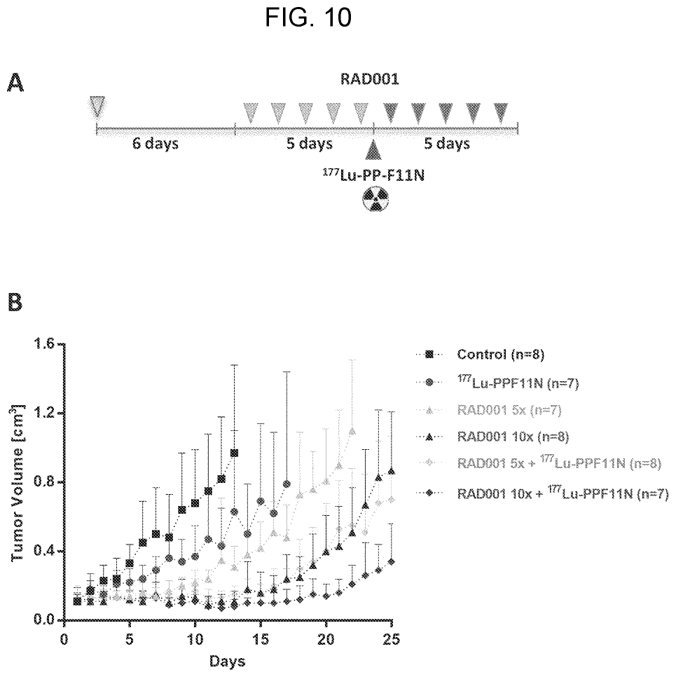

To evaluate the therapeutic effects of the combinatory treatment, the tumor growth and the mean survival time of immunocompromised A431/CCKBR-tumor bearing nude mice were investigated after the administration of 5 or 10 doses of RAD001 alone, or in combination with ¹⁷⁷Lu-PP-F11N as indicated in FIG. 10A.

### Animal Therapeutic Study:

For the animal study, human epidermoid carcinoma A431 cell line, which overexpresses CCKBR, was generated previously and kindly provided by Dr. Luigi Aloj. A431/CCKBR cells were cultured in DMEM, supplemented with 10% FCS, 2 mM glutamine and antibiotics (0.1 mg/mL streptomycin, 100 IU penicillin) at 37° C. and 5% CO2. Prior to tumor implantation, 5×10⁶ of A431/CCKBR cells in 0.1 mL of phosphate-buffered saline (PBS) containing 0.9% NaCl were injected subcutaneously into CD-1 female nude mice (Charles Rivers, Germany) anesthetized by isoflurane/oxygen inhalation. After 6 days, animals were randomly distributed into experimental groups and tumor size was measured non-invasively with a caliper. RAD001 (3 mg/kg), or PBS (control) were administered via intraperitoneal injection daily for 5 or 10 days, as indicated. HPLC-purified 60 MBq of ¹⁷⁷Lu-PPF11N in 100 μL PBS was injected intravenously, whereas the control group was injected with 100 μL PBS. The tumor diameters and mice weight were recorded daily. The tumor volume was calculated by using the formula V=(W2×L)/2. The nude mice were sacrificed when the tumor volume exceeded 1.5 cm³. In addition, the unwell mice (total n=2) or mice with ulcerated tumors (total n=7), present in all groups, were sacrificed prematurely. These mice were excluded from the analysis. All experiments were performed in accordance with Swiss Animal Protection Laws.

In this experiment, GraphPad Prism 7.00 for Windows was used for all statistical analysis. One-way ANOVA combined with two-stage linear step-up procedure of Benjamini, Krieger and Yekutieli for multiple comparison test was used for all treated groups. Log-rank (Mantel-Cox) test was performed to compare different survival curves of the treatment groups with the control group. Endpoints were defined as death in the survival curves. Values of P≤0.05 were considered statistically significant.

### Results:

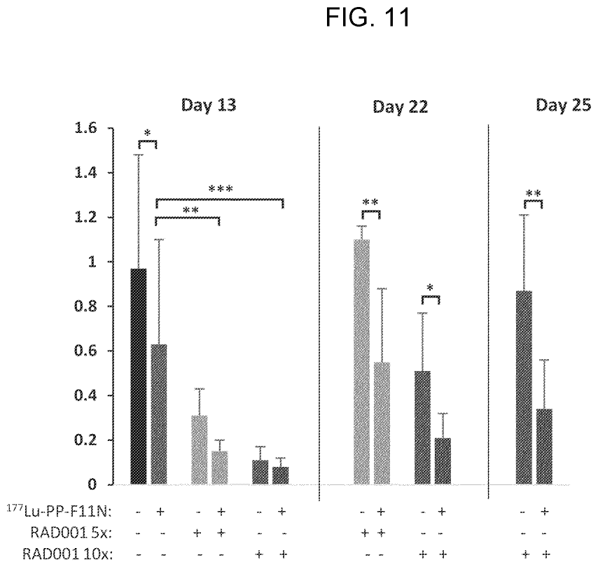

Both RAD001 and ¹⁷⁷Lu-PP-F11N single treatment as well as their combination significantly inhibited tumor growth (FIG. 10B). On day 13, where all mice were still present in all groups, the average tumor volume in the control group reached 0.97 cm³. The average tumor size in mice treated with ¹⁷⁷Lu-PP-F11N, 5, 10 doses of RAD001, and 5 or 10 doses of RAD001 in combination with ¹⁷⁷Lu-PP-F11N were significantly reduced (P<0.05) as compared to the control group and reached 0.63, 0.31, 0.11 and 0.15 or 0.08 cm³, respectively (FIG. 11). The tumor growth in 5× or 10×RAD001 and ¹⁷⁷Lu-PP-F11N-treated mice was significantly reduced as compared to monotherapy with ¹⁷⁷Lu-PP-F11N.

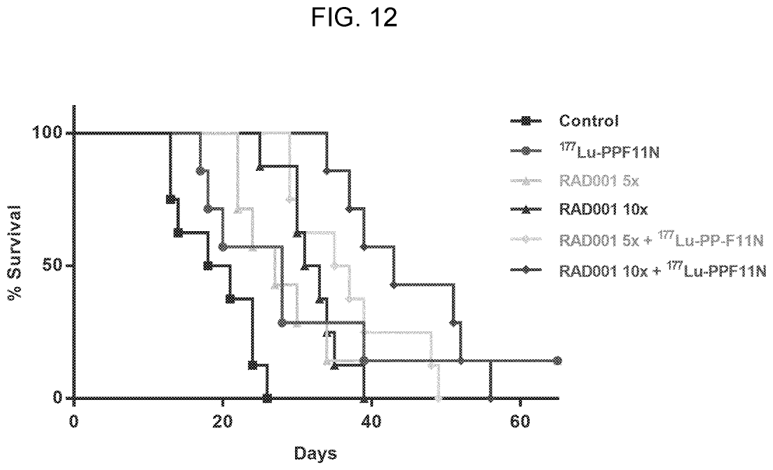

On day 22, the average tumor size in concomitant treated mice with 5 or 10 doses of RAD001 and ¹⁷⁷Lu-PP-F11N was significantly reduced as compared to the group, which received 5 or 10 doses of RAD001 only, respectively. On day 25, the average tumor size in concomitant treated mice with 10 doses of RAD001 and ¹⁷⁷Lu-PP-F11N was significantly reduced as compared to the monotherapy with 10 doses of RAD001. As shown by the Kaplan-Meier curves in FIG. 12 all treatments increased the life-span as compared to control group.

The median survival time in the control group was 19.5 days, whereas the median survival in the mice treated with ¹⁷⁷Lu-PP-F11N, 5 or 10 doses of RAD001 and 5 or 10 doses of RAD001 in combination with ¹⁷⁷Lu-PP-F11N was extended to 28, 27, 32, 36 and 43 days, respectively, as shown in table 1 below.

TABLE 1
Extended median survival times in treated
groups compared with control.

| | Median Survival |
|---|---|
| Control | 19.5 |
| ¹⁷⁷Lu-PP-F11N | 28* |
| RAD001 5x | 27* |
| RAD001 10x | 32*** |
| RAD001 5x + ¹⁷⁷Lu-PP-F11N | 36*** |
| RAD001 10x + ¹⁷⁷Lu-PP-F11N | 43*** |

*P < 0.05,
**P < 0.01,
***P < 0.001.

During therapy, there was a constant increase in the body weight and no significant differences between control and treated mouse groups (data not shown).

1-21. (canceled)

22. A composition, comprising:
(i) rapamycin and/or a rapalog; and
(ii) a radiolabeled gastrin analogue.

23. The composition according to claim 22, wherein the rapalog is a compound selected from the group consisting of: Everolimus (RAD001), Temsirolimus (CCI-779), Ridaforolimus (AP-23573), and combinations thereof.

24. The composition according to claim 22, wherein the radiolabeled gastrin analogue has the following formula (1):
(1)
(SEQ ID NO: 1)
Y-S-Axx-Bxx-Cxx-Gly-Trp-Dxx-Asp-Phe-NH₂
wherein,
Axx represents an amino acid selected from Glu, D-Glu, Ala, Asp, Gln, Lys, His, Arg, Ser and Asn;
Bxx represents an amino acid selected from Ala, Ser, Val, Cys, Thr and Gly;
Cxx represents an amino acid selected from Tyr, Ala, Phe, Trp and His;
Dxx represents an amino acid isosteric with methionine selected from norleucine (Nle), 2-amino-5-heptenoic acid, homo-norleucine (homo-Nle), 2-amino-4-methoxybutanoic acid, telluro-methionine (Te-Met), seleno-methionine (Se-Met) and phenylglycine (Phg);
S is a spacer containing at least one an amino acid having a negative charge, Gln and D-Gln; and
Y represents a moiety that contains a radionuclide.

25. The composition according to claim 22, wherein the radiolabeled gastrin analogue has the following formula (2) (SEQ ID NO:9):
(2)
Y-DGlu-DGlu-DGlu-DGlu-DGlu-DGlu-Ala-Tyr-Gly-Trp-
Dxx-Asp-Phe-NH₂
wherein:
Dxx represents an amino acid isosteric with methionine; and
Y represents a moiety that comprises a radionuclide.

26. The composition according to claim 24, wherein:
Dxx is Nle; and/or
Y is 1,4,7,10-tetraazacyclododecane-1,4,7,10-tetraacetic acid (DOTA), 1,4,7-triazacyclononane-1,4,7-triacetic acid (NOTA) or 1,4,7-triazacyclononane,1-glutaric acid-4,7-acetic acid (NODAGA).

27. The composition according to claim 24, wherein the radionuclide is selected from the group consisting of: ¹⁸F, ¹²⁴I, ¹³¹I, ⁸⁶Y, ⁹⁰Y, ¹⁷⁷Lu, ¹¹¹In, ¹⁸⁸Re, ⁶⁴Cu, ⁶⁷Cu, ¹⁴⁹Tb, ¹⁶¹Tb, ⁸⁹Sr, ^{44/43}Sc, ⁴⁷Sc and ¹⁵³Sm.

28. The composition according to claim 24, wherein the radionuclide is selected from the group consisting of: ¹⁷⁷Lu, ⁹⁰Y and ¹¹¹In.

29. The composition according to claim 24, wherein the radionuclide is ¹⁷⁷Lu.

30. The composition according to claim 22, wherein:
the rapalog is Everolimus (RAD001); and
the radiolabeled gastrin analogue has the following formula (3) (SEQ ID NO: 10):
(3)
DOTA-DGlu-DGlu-DGlu-DGlu-DGlu-DGlu-Ala-Tyr-Gly-
Trp-Nle-Asp-Phe-NH₂
wherein the DOTA chelates ¹⁷⁷Lu.

31. The composition according to claim 22, wherein the (i) rapamycin and/or rapalog and (ii) the radiolabelled gastrin analogue are formulated in separated dosage forms, which may be administered simultaneously and/or sequentially, and are co-packaged, or co-presented in separate packaging.

32. Kit-of-parts, comprising:
(i) rapamycin and/or a rapalog; and
(ii) a radiolabeled gastrin analogue.

33. The kit-of-parts according to claim 32, wherein (i) the rapalog is a compound selected from the group consisting of: Everolimus (RAD001), Temsirolimus (CCI-779), Ridaforolimus (AP-23573), and combinations thereof.

34. A combination product, comprising:
(i) rapamycin and/or a rapalog; and
(ii) a radiolabeled gastrin analogue.

35. The combination product according to claim 34, wherein the radiolabelled gastrin analogue has the following formula (1):
(1)
(SEQ ID NO: 1)
Y-S-Axx-Bxx-Cxx-Gly-Trp-Dxx-Asp-Phe-NH₂
wherein,
Axx represents an amino acid selected from Glu, D-Glu, Ala, Asp, Gln, Lys, His, Arg, Ser and Asn;
Bxx represents an amino acid selected from Ala, Ser, Val, Cys, Thr and Gly;
Cxx represents an amino acid selected from Tyr, Ala, Phe, Trp and His;
Dxx represents an amino acid isosteric with methionine selected from norleucine (Nle), 2-amino-5-heptenoic acid, homo-norleucine (homo-Nle), 2-amino-4-methoxybutanoic acid, telluro-methionine (Te-Met), seleno-methionine (Se-Met) and phenylglycine (Phg);
S is a spacer containing at least one an amino acid having a negative charge, Gln and D-Gln; and
Y represents a moiety that contains a radionuclide.

36. A method of treating cholecystokinin B (CCKB) receptor positive cancer or tumors, which comprises the steps of:
providing one of a composition having (i) rapamycin and/or rapalog and (ii) radiolabeled gastrin analogue, kit-of-parts having (i) the rapamycin and/or the rapalog and (ii) the radiolabeled gastrin analogue, or a combination product having (i) the rapamycin and/or the rapalog and (ii) the radiolabeled gastrin analogue; and
performing one of:
administering (i) the rapamycin and/or the rapalog along with (ii) the radiolabeled gastrin analogue from one of the composition, the kit-of-parts or the combination product; or
administering (i) the rapamycin and/or the rapalog before administering (ii) the radiolabeled gastrin analogue from one of the composition, the kit-of-parts or the combination product.

37. The method according to claim 36, wherein the (i) rapamycin and/or the rapalog is administered up to two months before (ii) the radiolabeled gastrin analogue.

38. The method according to claim 36, which further comprises administering (i) the rapamycin and/or the rapalog once daily over 1 to 14 consecutive days, before (ii) the radiolabeled gastrin analogue is administered.

39. The method according to claim 36, wherein the CCKB receptor positive cancer or tumors is/are selected from medullary thyroid cancer (MTC), gliomas, gastroenteropancreatic neuroendocrine tumors (GEP-NETs), astrocytomas, stomach cancer, colon cancer, ovarian cancer, breast cancer, and any CCKB receptor positive tumors or cancer.

40. The method according to claim 36, wherein the CCKB receptor positive cancer or tumors is from medullary thyroid cancer (MTC).

41. The method according to claim 36, which further comprises administering (i) the rapamycin and/or rapalog or (ii) the radiolabeled gastrin analogue concurrently with, before or after at least one other therapeutic agent or therapy such as chemotherapeutic agents or immunomodulatory agents.

42. A method for treating cholecystokinin B (CCKB) receptor positive cancer or tumors, which comprises the steps of:
providing one of a composition having (i) rapamycin and/or rapalog and (ii) radiolabeled gastrin analogue, kit-of-parts having (i) the rapamycin and/or the rapalog and (ii) the radiolabeled gastrin analogue, or a combination product having (i) the rapamycin and/or the rapalog and (ii) the radiolabeled gastrin analogue; and
administering a therapeutically effective amount of the (i) the rapamycin and/or the rapalog along with (ii) the radiolabeled gastrin analogue from one of the composition, the kit-of-parts or the combination product to a patient in need thereof.


SEQUENCE LISTING (11 sequences)

SEQ ID NO: 1 - protein, 9 aa, Artificial Sequence
  Polypeptide
  VARIANT (1)..(1): Spacer comprising at least one amino acid containing a negative charge, Gln and D-Gln, preferably a tetra- or pentapeptide comprising at least one amino acid selected from Glu, D-Glu, beta-glutamic acid (beta-Glu), Gln, D-Gln, Asp and D-Asp
  VARIANT (2)..(2): Xaa can be Glu, D-Glu, Ala, Asp, Gln, Lys, His, Arg, Ser and Asn, preferably Glu or D-Glu
  VARIANT (3)..(3): Xaa can be  Ala, Ser, Val, Cys, Thr and Gly, preferably Ala or Ser
  VARIANT (4)..(4): Xaa can be  Tyr, Ala, Phe, Trp and His, preferably Tyr
  VARIANT (7)..(7): Xaa can be an amino acid isosteric with methionine, preferably norleucine, 2-amino-5-heptenoic acid, homo-norleucine (homo-Nle), 2-amino-4-methoxybutanoic acid, telluro-methionine (Te-Met), seleno-methionine (Se-Met) and phenylglycine (Phg)
  MOD_RES (7)..(7): Xaa can be an amino acid isosteric with methionine, preferably norleucine, 2-amino-5-heptenoic acid, homo-norleucine (homo-Nle), 2-amino-4-methoxybutanoic acid, telluro-methionine (Te-Met), seleno-methionine (Se-Met) and phenylglycine (Phg)
XXXXGWXDF

SEQ ID NO: 2 - protein, 13 aa, Artificial Sequence
  Polypeptide
LEEEEEAYGWMDF

SEQ ID NO: 3 - protein, 5 aa, Artificial Sequence
  Polypeptide
  VARIANT (3)..(3): Xaa can be Met or any amino acid isosteric with Met such as norleucine
  MOD_RES (3)..(3): Nle
GWXDF

SEQ ID NO: 4 - protein, 8 aa, Artificial Sequence [sequence omitted]

SEQ ID NO: 5 - DNA, 21 bp, Artificial Sequence
  Polynucleotide
  misc_feature (1)..(19): RNA
  misc_feature (20)..(21): DNA
uauacgaguaguagcaccatt

SEQ ID NO: 6 - DNA, 21 bp, Artificial Sequence
  Polynucleotide
  misc_feature (1)..(19): RNA
  misc_feature (20)..(21): DNA
ccgccaaaggauggaguactt

SEQ ID NO: 7 - DNA, 21 bp, Artificial Sequence
  Polynucleotide
  misc_feature (1)..(19): RNA
  misc_feature (20)..(21): DNA
cguacgcggaauacuucgatt

SEQ ID NO: 8 - protein, 13 aa, Artificial Sequence
  Polypeptide
  VARIANT (11)..(11): Xaa can be any amino acid isosteric with methionine
EEEEEEAYGWXDF

SEQ ID NO: 9 - protein, 13 aa, Artificial Sequence
  Polypeptide
  VARIANT (11)..(11): Xaa can be any amino acid isosteric with methionine such as norleucine (Nle), 2-amino-5-heptenoic acid, homo-norleucine (homo-Nle), 2-amino-4-methoxybutanoic acid, telluro-methionine (Te-Met), seleno-methionine (Se-Met) or Phg
  MOD_RES (11)..(11): Xaa can be any amino acid isosteric with methionine such as norleucine (Nle), 2-amino-5-heptenoic acid, homo-norleucine (homo-Nle), 2-amino-4-methoxybutanoic acid, telluro-methionine (Te-Met), seleno-methionine (Se-Met) or Phg
EEEEEEAYGWXDF

SEQ ID NO: 10 - protein, 13 aa, Artificial Sequence
  Polypeptide
  MOD_RES (11)..(11): Nle
EEEEEEAYGWXDF

SEQ ID NO: 11 - protein, 5 aa, Artificial Sequence
  Polypeptide
  VARIANT (3)..(3): Xaa can be Met or any amino acid isosteric with Met
GWXDF